(19) United States
(12) Patent Application Publication
(10) Pub. No.: US 2025/0270487 A1
(43) Pub. Date: Aug. 28, 2025

(54) CONTAINER ASSEMBLY FOR MICROBIOREACTOR

(71) Applicant: Beckman Coulter, Inc., Brea, CA (US)

(72) Inventors: Alexander Kremers, Jülich (DE); David Meyersieck, Köln (DE); Simon Sattler, Aachen (DE)

(73) Assignee: Beckman Coulter, Inc., Brea, CA (US)

(21) Appl. No.: 18/559,303

(22) PCT Filed: May 6, 2022
(86) PCT No.: PCT/US2022/028201

§ 371 Date: Nov. 6, 2023

Related U.S. Application Data
(60) Provisional application No. 63/185,650, filed on May 7, 2021.
(60) Provisional application No. 63/227,210, filed on Jul. 29, 2021.

(51) Int. Cl. C12M 1/32; C12M 1/00; C12M 1/12; C12M 1/34; C12M 3/00

(52) U.S. Cl. CPC C12M 23/12; C12M 23/34; C12M 23/38; C12M 23/42; C12M 29/20; C12M 37/04; C12M 41/34

(57) ABSTRACT

A gassing lid assembly enables gas-tight sealing of sample containers in general, also referred to as microplates in some embodiments, with simultaneous guided access for the pipetting unit of a dispensing/pipetting robot, also referred to as a pipettor. The component enables both gas-tight sealing and guided access for the pipetting robot. The gassing lid serves a number of purposes at the same time and provides the following advantages in a non-limiting fashion: a gas tight seal, robot integration without a gassing lid, robot integration with a gassing lid, a sealing mechanism, and anaerobic transport. Reducing the volume above reservoirs of a sample container (e.g., the volume above wells of a microplate) is advantageous in that it reduces the safety risk of high concentrations of gases such as oxygen.

### BACKGROUND

This application is a U.S. National Stage Application of PCT/US2022/028201, filed on May 6, 2022, which claims the benefit of and priority to U.S. Provisional Patent Application Nos. 63/185,650, filed May 7, 2021, and 63/227,210, filed Jul. 29, 2021, the disclosures of which are incorporated by reference herein in their entireties.

In many areas of biology, pharmacology, and medicine, biological systems are screened for the selection of suitable biological strains, enzymes, or suitable culture media and culture conditions, among other examples. In this context, there is a need for high sample throughputs which may be achieved via parallelization of experiments.

A microplate, or microtiter plate, is a flat plate with multiple wells that are used as small test tubes, and is one example of a device that can be utilized to achieve a high number of parallel operations. As an illustrative example, each of the individual wells may be filled with a medium, inoculated to introduce cells into the medium, and incubated at a particular temperature using a shaking incubator. Process parameters, such as a pH value, concentrations of dissolved oxygen (DO), dissolved carbon dioxide, and biomass, among other parameter values, may be continuously measured for each individual well during the growth process.

Miniaturization and parallelization in the industrial production of microorganisms have gained in economic importance in recent decades. One challenge in the cultivation of microorganisms is real-time monitoring of the process parameters of the cell cultures being produced. Controlling the supply of nutrients and the pH, and monitoring the biomass growth and the DO, allows parallel optimization of cell cultures in miniaturized bioreactors to maximize the yield of active substances, vitamins, peptides or proteins.

### SUMMARY

In general terms, the present disclosure relates to a container assembly for a microbioreactor. In one configuration, the container assembly provides an improved seal between a sample container and a gassing lid that allows a pipette tip to be inserted into a well of the sample container during agitation inside the microbioreactor. Various aspects are described in this disclosure, which include, but are not limited to, the following aspects.

One aspect relates to a component which enables a gas-tight sealing of sample containers. In some examples, the component enables gas-tight sealing of microplates.

In some further examples, in addition to enabling gas-tight sealing of sample containers, the component simultaneously provides guided access for a pipette tip. The component that enables both gas-tight sealing and guided access for the pipette tip is sometimes referred to from here on out as the “gassing lid” or “lid housing” or “lid assembly”. The gassing lid serves a number of purposes at the same time and provides the following advantages in a non-limiting fashion: a gas tight seal, robotic integration, and anaerobic transport.

The gassing lid can significantly reduce the headspace volume above the wells of a sample container (e.g., the wells of a microplate). Reducing this volume is advantageous in that it reduces the safety risk of high concentrations of gases such as oxygen.

The gassing lid provides the several advantages including: reduced headspace in the sample container safely allowing higher O₂ concentrations in the sample container: guide elements that help guide insertion of a pipette tip while agitating the sample container: multiple resilient layers with slits that open when pipette tip inserted and that close when the pipette tip is removed: sealing surfaces that distribute pressure optimally around edges of the sample container and gassing lid and allowing for partitions: and a seal that allows the sample container to be transported as a single unit for anaerobic cultivation in an aerobic workspace. Also, the gassing lid allows anaerobic cultivation inside a microbioreactor because the gassing lid prevents oxygen from entering into the cultivation wells of the sample container while in the microbioreactor.

In some examples, there are at least two types of gassing lids. A first type of gassing lid is compatible with microfluidic microplates that have microfluidics in the well bottoms coupling liquid reagents disposed in a first set of wells to a second set of wells (which have cells for cultivation). These lids include a partition separating the two sets of wells. Although the disclosure describes this first type of gassing lid as having a single partition that separates the wells into two sets, any suitable number of sets of wells separated by any suitable number of partitions are contemplated by this disclosure.

A second type of gassing lid is compatible non-microfluidic microplates (or standard microplates). These microplates do not have microfluidics for the wells.

Both microfluidic and non-microfluidic microplates allow feeding and pH control to take place simultaneously during direct nitrogen (e.g., 100% N₂) gassing of the sample container with adjustable flowrates such as, for example, between 5-50 mL/min.

Another aspect relates to enabling anaerobic or microaerophilic cultivation, sampling, feeding, and pH control when the microplates are in an aerobic environment.

### BRIEF DESCRIPTION OF THE DRAWINGS

The following drawing figures, which form a part of this application, are illustrative of the described technology and are not meant to limit the scope of the disclosure in any manner.

FIG. 1 is an isometric view of a microbioreactor.

FIG. 2 is a top isometric view of a container assembly that fits inside a cultivation chamber of the microbioreactor of FIG. 1.

FIG. 3 is a bottom isometric view of the container assembly.

FIG. 4 is an exploded isometric view of the container assembly.

FIG. 5 is an exploded front elevation view of the container assembly.

FIG. 6 is a cross-sectional view of the container assembly.

FIG. 7 is a detailed view of a resilient layer of the container assembly.

FIG. 8 is a top view of an example of a sample container that includes a plurality of wells, the sample container being a component of the container assembly of FIG. 2.

FIG. 9 is a bottom view of a first example of a lid housing of the container assembly.

FIG. 10 is a bottom isometric view of the lid housing shown in FIG. 9.

FIG. 11 is a bottom isometric view of the lid housing and a sample container.

FIG. 12 is a bottom view of another example of the lid housing.

FIG. 13 is a cross-sectional view of the container assembly of FIG. 2 showing a pipette tip inserted through a guide element of the lid housing.

FIG. 14 is a cross-sectional view of the container assembly of FIG. 2 after the pipette tip has been removed from the container assembly.

FIG. 15 is a cross-sectional view of a sealing mechanism of the container assembly, the sealing mechanism being shown in an opened position.

FIG. 16 is another cross-sectional view of the sealing mechanism of FIG. 15, the sealing mechanism being shown in a closed position.

FIG. 17 is a cross-sectional view showing a seal between the lid housing and sample container of the container assembly of FIG. 2.

FIG. 18 is a cross-sectional view showing a release pin of the lid housing.

FIG. 19 is a top isometric view showing an example of the container assembly positioned on top of a base of the microbioreactor of FIG. 1.

FIG. 20 is a cross-sectional view of the container assembly of FIG. 19 on the base.

FIG. 21 is a top isometric view of the base FIG. 19.

FIG. 22 is a top isometric view showing another example of the container assembly positioned on top of a base of the microbioreactor of FIG. 1.

FIG. 23 is a cross-sectional view of the container assembly of FIG. 22 on the base.

FIG. 24 is a top isometric view of the base of FIG. 22.

FIG. 25 schematically shows an example of a computer control system of the microbioreactor of FIG. 1.

FIG. 26 is an isometric view of a mechanical system of the microbioreactor of FIG. 1 for positioning an optical sensor under the container assembly of FIG. 2.

FIG. 27 is an isometric view of a light-emitting diode array module (LAM) that can be used to illuminate the cultivation chamber of the microbioreactor of FIG. 1.

FIG. 28 is a bottom isometric view of the LAM mounted underneath the microbioreactor of FIG. 1.

FIG. 29 is a schematic diagram of the LAM.

FIG. 30 is an isometric view of a cooling plate of the LAM.

FIG. 31 is a bottom isometric view of an example of a lid housing that is adapted to cool the sample container.

FIG. 32 is a top isometric view of the lid housing of FIG. 31.

FIG. 33 illustrates a microfluidic valve configuration.

FIG. 34 illustrates an example of a method of anaerobic cultivation that can be performed using the container assembly.

FIG. 35 illustrates another example of a method of anaerobic cultivation that can be performed using the container assembly.

FIG. 36 is a graph showing dissolved oxygen, biomass gain, and added feed solution over cultivation time during a cultivation process inside the container assembly.

FIG. 37 is a graph showing pH and added NaOH volume over cultivation time during a cultivation process inside the container assembly.

FIG. 38 is a graph showing biomass over cultivation time during a cultivation process inside the container assembly.

FIG. 39 is a graph showing oxygen concentration, pH signal, and added NaOH volume over cultivation time during a cultivation process inside the container assembly.

FIG. 40 is a graph showing biomass and added feed volume over cultivation time during a cultivation process inside the container assembly

FIG. 41 is a graph showing pH, oxygen concentration, and added volume of NaOH over cultivation time during a cultivation process inside the container assembly.

### DETAILED DESCRIPTION

Throughout all the figures, same or corresponding elements may generally be indicated by same reference numerals. These depicted embodiments are to be understood as illustrative of the invention and not as limiting in any way. It should also be understood that the figures are not necessarily to scale and that the embodiments may be illustrated by graphic symbols, phantom lines, diagrammatic representations and fragmentary views. In certain instances, details which are not necessary for an understanding of the present invention or which render other details difficult to perceive may have been omitted.

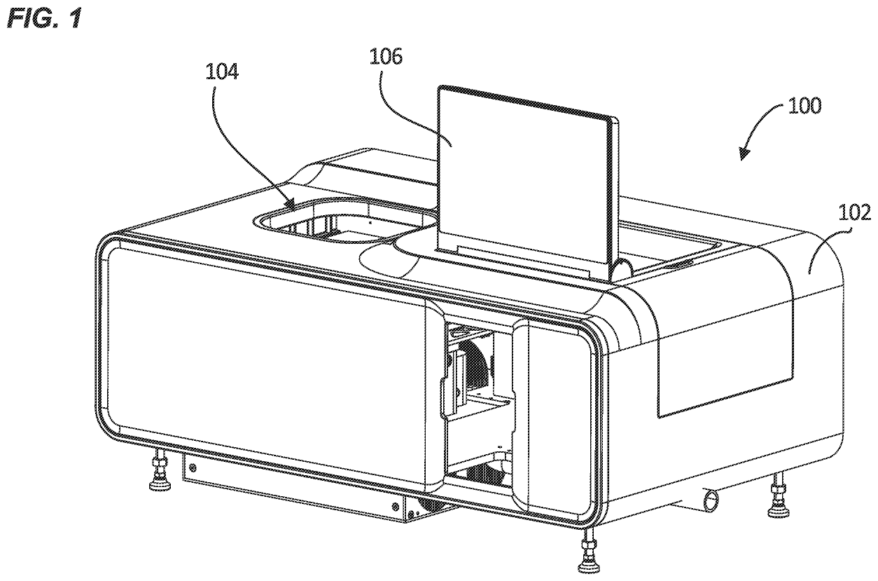

FIG. 1 is an isometric view of an example of a microbioreactor 100. As shown in FIG. 1, the microbioreactor 100 includes a housing 102 that defines a cultivation chamber 104. The microbioreactor 100 measures parameters such as biomass, pH, dissolved oxygen (DO), and fluorescence online while running a cultivation inside the cultivation chamber 104. Additionally, the microbioreactor 100 includes a touchscreen display 106 that allows a user to control the shaking speed, temperature, gas concentration, gas flow rate, and humidity inside the cultivation chamber 104. Alternatively or additionally, the microbioreactor 100 may be communicatively coupled to a separate computing device that may allow for such control.

In some aspects, the microbioreactor 100 can share similar components, features, and functionalities with the microreactors described in U.S. Pat. No. 8,268,632, titled Method and Device for Recording Process Parameters of Reaction Fluids in Several Agitated Microreactors, issued on Sep. 18, 2012, U.S. Pat. No. 8,828,337, titled Microreactor, issued on Sep. 9, 2014, U.S. Pat. No. 8,932,544, titled Microreactor Array, Device Comprising a Microreactor Array, and Method for Using a Microreactor Array, issued on Jan. 13, 2015, and U.S. Pat. No. 10,421,071, titled Microreactor System, issued on Sep. 24, 2019, the entireties of which are hereby incorporated by reference.

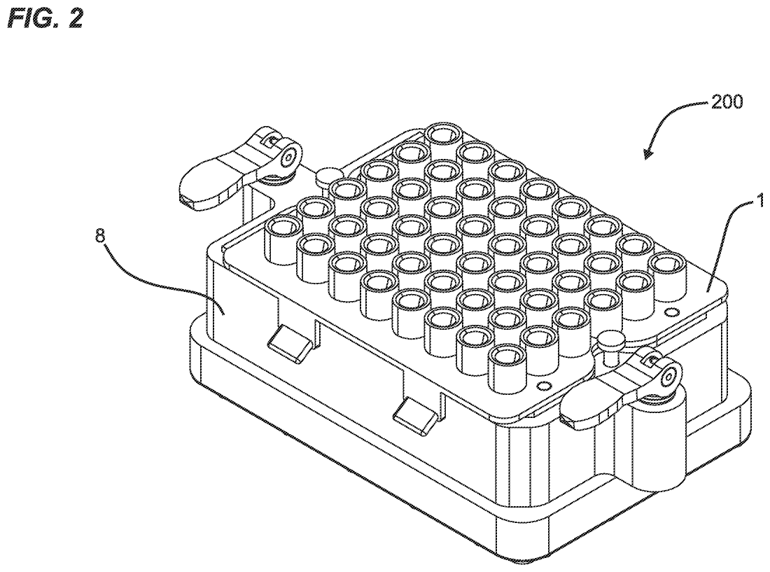

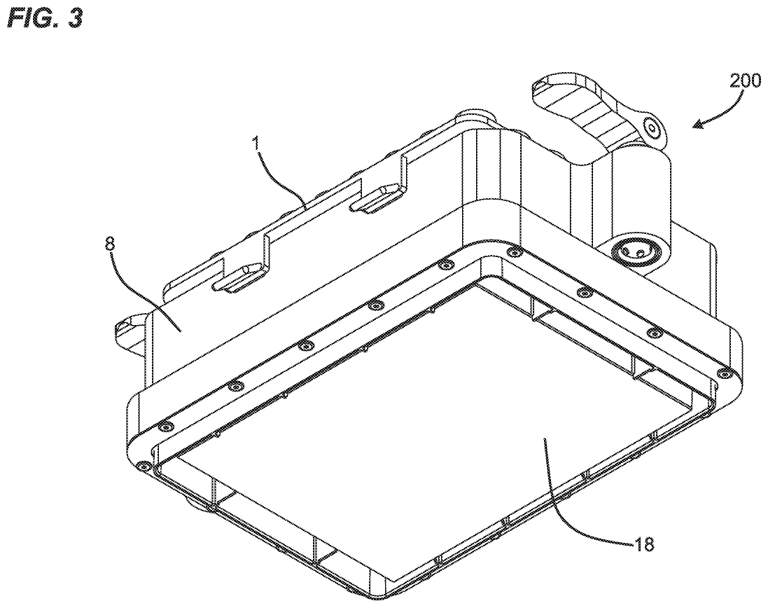

FIGS. 2 and 3 are isometric views of a container assembly 200 that fits inside the cultivation chamber 104 of the microbioreactor 100. The container assembly 200 includes a lid housing 8 that can attach or otherwise be coupled to a sample container 18. In some examples, the sample container 18 is a microplate or microtiter plate. The lid housing 8 seals the sample container 18 in a gastight manner. The lid housing 8 allows safety-critical gases to be fed into and discharged from the sample container 18 in any concentration and at any flow rate.

The container assembly 200 provides advantages that include at least a gastight seal of the sample container 18. The gastight seal of the sample container 18 enables the controlled introduction and discharge of safety-critical gases without the gases coming into contact with the atmosphere of the cultivation chamber 104 and other components of the microbioreactor 100. This enables a high level of control over gas concentrations in a headspace above the sample container 18. The headspace is the space between the sample container 18 and a bottom interior surface 28 of the lid housing 8. Furthermore, this enables maintaining high concentrations of oxygen or other gases that are unsafe (e.g., gases that are combustible, toxic, able to asphyxiate, etc.) within the container assembly 200 during cultivation and reducing safety risks such as fire or explosion. For example, the container assembly 200 allows for maintaining up to 100% pure oxygen in the headspace under reduced safety risks. Additionally, the container assembly 200 can further significantly reduce the headspace above the sample container 18 as compared to conventional systems. This further contributes to reducing the safety risks posed by, for example, high concentrations of combustible gases like oxygen by reducing the overall volume of such gases. Also, the design and selection of materials for the cultivation chamber 104 are no longer constrained by having to account for direct contact with critical gases, which reduces the technical effort required to build the microbioreactor 100.

Since the gases are fed into and discharged from the sample container 18 in a controlled manner, the flow of gases with asphyxiation potential, such as N₂ and CO₂, can be increased as needed. Additionally, the lid housing 8 reduces energy and gas consumption because only a headspace above the sample container 18 has to be humidified and gassed, rather than the entirety of the cultivation chamber 104 of the microbioreactor 100.

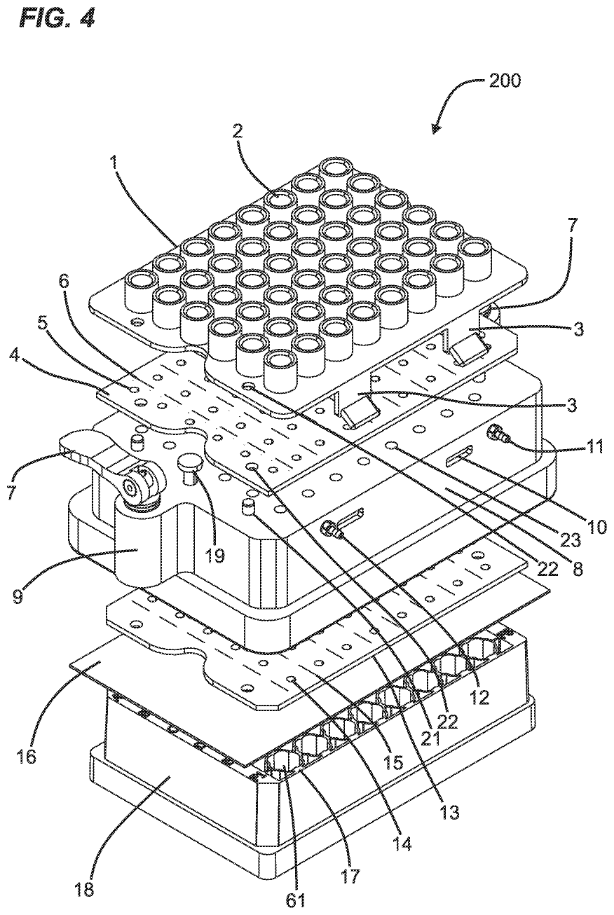

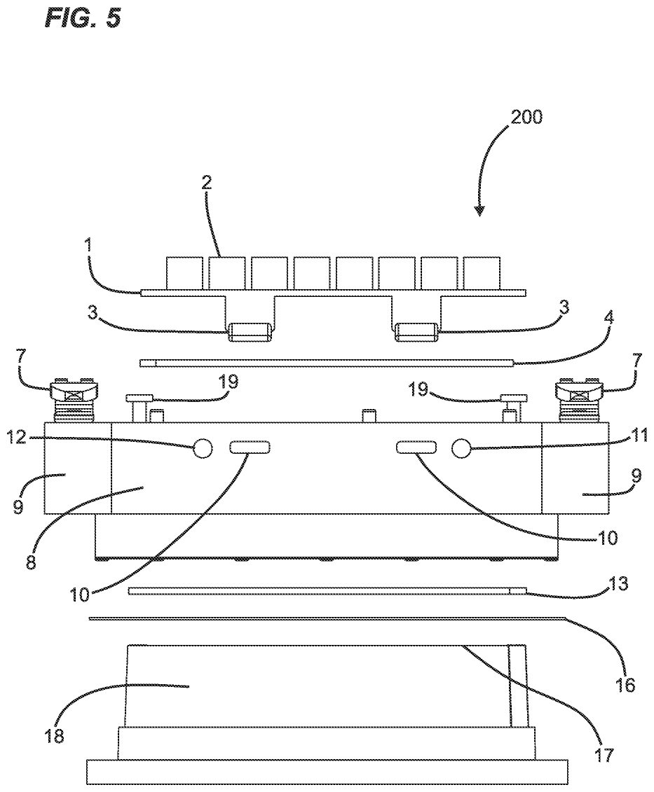

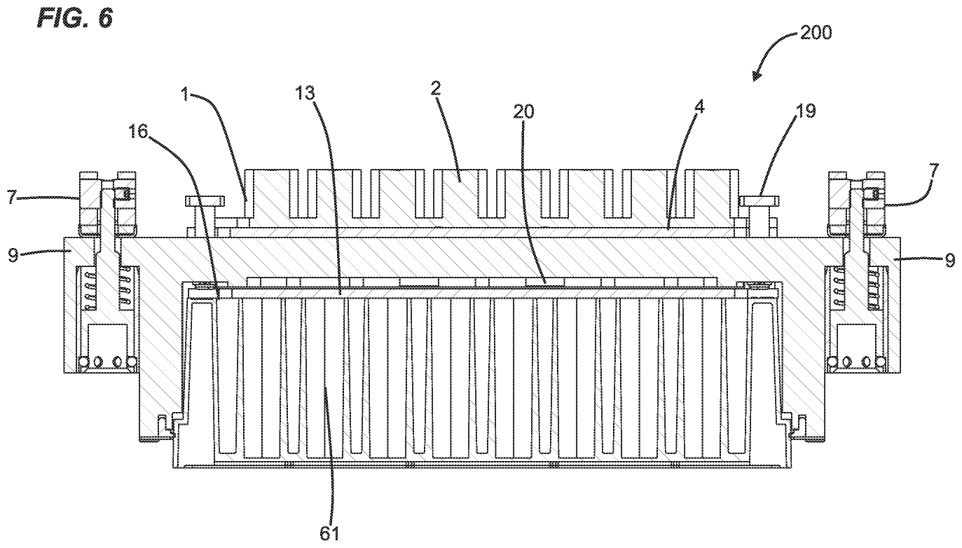

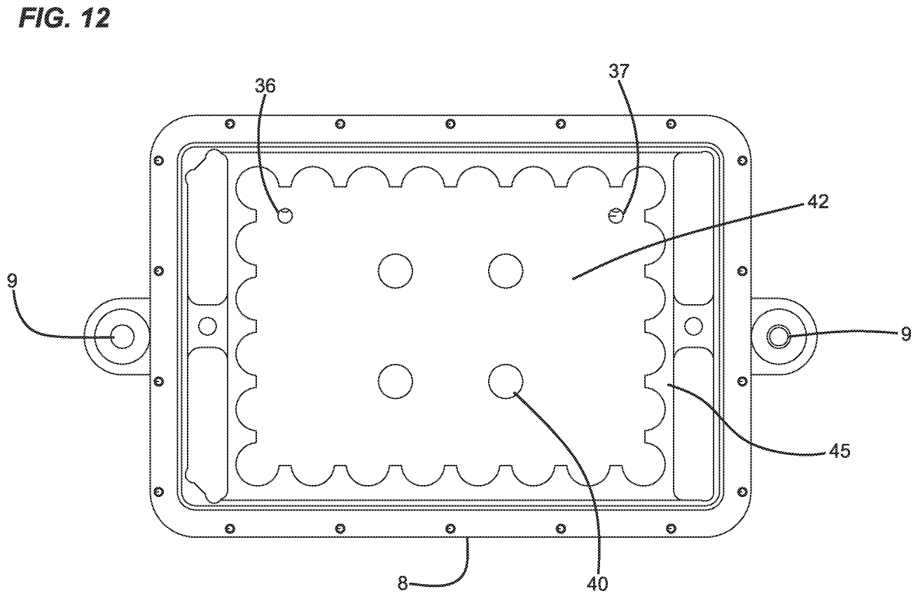

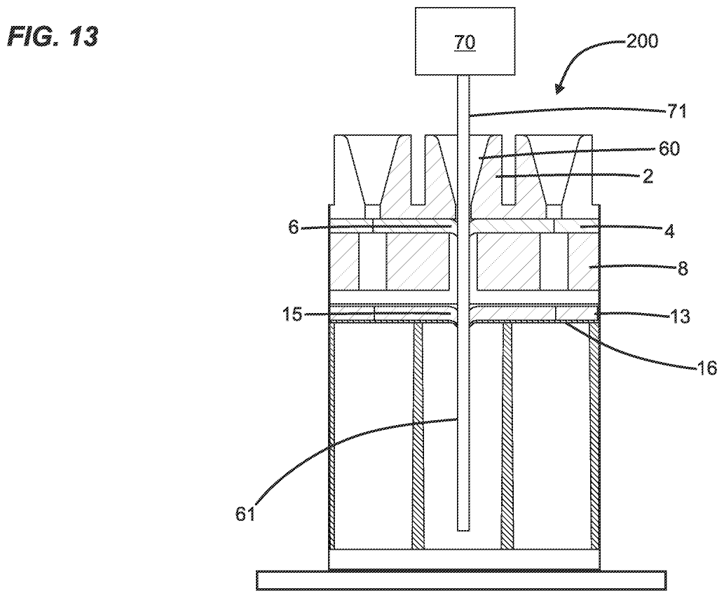

FIG. 4 is an exploded isometric view of the container assembly 200. FIG. 5 is an exploded front elevation view of the container assembly 200. FIG. 6 is a cross-sectional view of the container assembly 200. Referring now to FIGS. 2-6, the sample container 18 includes rows of wells 61 that are each configured to separately contain a cell culture or reagent. The sample container 18 is completely covered by the lid housing 8, but is still accessible for one or more pipette tips (see pipette tip 71 shown in FIG. 13) to feed liquids into the wells 61 or to take probes from the wells 61. Thus, the lid housing 8 has an assembly which allows the pipette tip 71 to enter the container assembly 200 without allowing the gas atmosphere to escape from the container assembly 200. The lid housing 8 has through-holes 23 above each of the wells 61 for the pipette tip 71 to access each well.

A guide structure 1 is releasably and reversibly coupled to the lid housing 8. The guide structure 1 includes a plurality of guide elements 2. The guide structure 1 further includes one or more attachment mechanisms 3 that are structured to engage corresponding slots 10 on the lid housing 8 to removably attach the guide structure 1 onto the lid housing 8.

The attachment mechanisms 3 are structured to flex against an exterior surface of the lid housing 8, and snap fit into the corresponding slots 10. The attachment mechanisms 3 can include handles that allow a user to disengage the attachment mechanisms 3 from the corresponding slots 10, and to thereby release the guide structure 1 from the lid housing 8. In alternative examples, the guide elements 2 form an integral part of the lid housing 8.

A first resilient layer 13 is positioned between the lid housing 8 and a sterile layer 16. A second resilient layer 4 is positioned between the lid housing 8 and the guide structure 1.

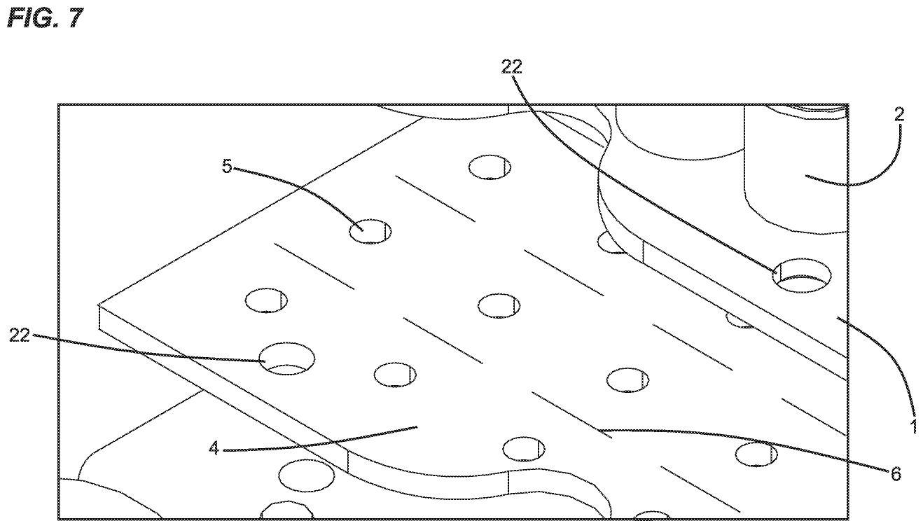

FIG. 7 is a detailed view of the second resilient layer 4. While the figures show the first and second resilient layers 13, 4 as being identical, in some examples they are not. Referring now to FIGS. 4 and 7, the first and second resilient layers 13, 4 each include apertures 15, 6. While the apertures 15, 6 are illustrated as slits having a linear shape, alternative shapes and configurations for the apertures 15,6 are possible.

As shown in FIG. 4, the apertures 6 of the second resilient layer 4 align with the through-holes 23, and the apertures 15 of the first resilient layer 13 align with the through-holes 23. The components of the container assembly 200 are arranged such that a pipette tip 71 can be inserted through a guide element 2, through an aperture 6 of the second resilient layer 4, through a through-hole 23 of the lid housing 8, and through an aperture 15 of the first resilient layer 13, such that the pipette tip 71 can pierce the sterile layer 16, and reach a well 61 of the sample container 18. In some examples, the guide element 2, the second resilient layer 4, the lid housing 8, and the first resilient layer 13 form a gassing lid for the container assembly 200.

At least the second resilient layer 4 includes holes 22 that align with pins 21 on a top exterior surface of the lid housing 8. Cooperation between the holes 22 on the second resilient layer 4 and the pins 21 allows the second resilient layer 4 to be fixed relative to the lid housing 8.

In some examples, the guide structure 1 includes holes 22 that align with the pins 21 on the top exterior surface of the lid housing 8. Cooperation between the holes 22 on the guide structure 1 and the pins 21 allows the guide structure 1 to be fixed relative to the lid housing 8.

At least the first resilient layer 13 includes holes 14 that allow for gases to pass through the first resilient layer 13. The holes 14 are positioned adjacent to the apertures 15. In the example depicted in the figures, the second resilient layer 4 also includes holes 5 that are adjacent to the apertures 6. For clarity, the holes 5 do not serve a purpose. The holes 5 are not aligned with the holes 14 of the first resilient layer 13, the through-holes 23 of the lid housing 8, or the guide elements 2 of the guide structure 1, and the holes 5 do not allow for gases to escape from the container assembly 200. Accordingly, the container assembly 200 is airtight. The holes 5 exist in the second resilient layer 4 only so that the same part can be manufactured for use as both the first and second resilient layers 13, 4. Accordingly, the holes 5 are optional. Thus, in alternative examples, the second resilient layer 4 does not include the holes 5.

The first and second resilient layers 13, 4 are made from a resilient material such as silicone. The resilient material of the first and second resilient layers 13, 4 can help reduce contamination and evaporation inside the container assembly 200, and maintain gas concentrations at desired levels by not allowing mixing within the container assembly 200.

The first and second resilient layers 13, 4 are resilient in that they are capable of recovering their size and shape after deformation. For example, the apertures 15, 6 are self-healing apertures that are configured to open when the pipette tip 71 (see FIG. 13) is inserted therethrough, and the apertures 15, 6 are configured to self-heal and close when the pipette tip 71 is removed therefrom, as will be discussed in more detail below.

It can be advantageous to use at least two resilient layers (i.e., the first and second resilient layers 13, 4) in the configuration described herein. For example, a bottom resilient layer (e.g., the first resilient layer 13) can keep the wells 61 in the sample container 18 covered for sterility and prevent evaporation. A top resilient layer (e.g., the second resilient layer 4) can provide additional sterility, and seals the top of the lid housing 8 (covering the through-holes 23 at all times when not pierced by a pipette tip) so as to help regulate the gas concentration in a headspace 20 (see FIG. 6) of the lid housing 8. In some examples, any number of additional layers may be used for added benefits (e.g., increased sterility and/or sealing).

The sterile layer 16 is made from a sterile material such as a cellulose membrane, or any other suitable layer that is biocompatible and capable of maintaining sterility. For example, the sterile layer 16 can be made from a fabric having a pore size that is small enough to not be permeable to microorganisms and water vapor, and that is large enough to be permeable to gases.

An adhesive can be used to secure the sterile layer 16 around a perimeter of the sample container 18. As an illustrative example, the adhesive can be applied to either the sterile layer 16 or the sample container 18 using an applicator or similar means. In some examples, the entire sterile layer 16 is an adhesive that can be directly applied onto the sample container 18.

The sterile layer 16 provides a sterile boundary between the sample container 18 and the cultivation chamber 104 of the microbioreactor 100. Advantageously, the cultivation chamber 104 does not have to be kept sterile at all times to prevent contamination of the cell cultures inside the wells 61 of the sample container 18. Additionally, the sterile layer 16 can reduce evaporation while also being permeable to gases such as O₂, N₂, CO₂, air, and the like.

The sterile layer 16 is useful in preventing or at least reducing contamination of cell cultures within the wells 61 of the sample container 18 (especially prior to first piercing by a pipette tip as described below), and is also useful in preventing or at least reducing evaporation from leaving the wells 61 of the sample container 18. As will be described further below, when taking a sample or adding suspending agents to the wells 61 of the sample container 18, the pipette tip 71 pierces the sterile layer 16 of the container assembly 200.

Although the holes that result from the pipette tip 71 piercing the sterile layer 16 may reduce some of the effects provided by the sterile layer 16 mentioned above, the reduction in effectiveness is mitigated in that the holes created from the pipette tip 71 piercing the sterile layer 16 are relatively small. Additionally, one or more resilient layers (e.g., the first and second resilient layers 13, 4 above the sterile layer 16) can help seal the headspace 20 above the wells 61 (e.g., sample reservoirs) in the sample container 18. The one or more resilient layers can also help reduce contamination after the sterile layer 16 is pierced, and further reduce evaporation.

Additionally, the one or more resilient layers provide an air-tight seal that allows for controlling and maintaining necessary gas concentrations in the headspace 20 above the wells 61 in the sample container 18. The lid housing 8 is configured to provide the headspace 20 above the wells 61 to allow gas exchange during cell cultivation. In some examples, the headspace 20 provided by the lid housing 8 can range from 20 mL to 400 mL. In some further examples, the headspace 20 provided by the lid housing 8 can range from 60 mL to 90 mL for a first type of gassing lid that has a partition and is configured to work with plates having microfluidics, as will be described further. In some further examples, the headspace 20 provided by the lid housing 8 can range from 80 to 120 mL for a second type of gassing lid that is configured to work with plates having no microfluidics and is therefore not partitioned, as will be described further.

As shown in FIGS. 4 and 5, the lid housing 8 can include gas ports 11, 12 that allow gas to enter and exit the headspace 20 above the wells 61 in the sample container 18. For example, the gas port 11 may be an inlet port and the gas port 12 may be an outlet port (or vice versa). In some examples, the gas inlet port may be coupled to a device that mixes two or more gases (e.g., oxygen, carbon dioxide, and nitrogen) to achieve a gas mixture having desired concentrations for supplying to the gas mixture to the headspace 20. The gas outlet port is used to exhaust gas from the headspace 20 at a desired flow rate (e.g., matching a flow rate of the gas inlet once a desired pressure has been achieved). Although not illustrated, in some examples, the lid housing can include multiple inlets for different gases. For example, it may include separate inlets for oxygen, carbon dioxide, and nitrogen.

The examples depicted in FIGS. 4 and 5 show the lid housing 8 as including two gas ports: a gas inlet port and a gas outlet port. In such examples, the lid assembly 8 can be used for non-microfluidic applications. In alternative examples, such as when the lid housing 8 is adapted for microfluidic applications, the lid housing 8 can include an additional gas port for introducing or removing a pressurizing gas to/from the space above reservoir wells (such space being partitioned from the headspace above cultivation wells) so as to control fluid flow of reagents from the reservoir wells to the cultivation wells, as will be described further below. Although this disclosure discloses a certain number of gas ports, any suitable number of gas ports is contemplated. For example, additional gas ports may be included for different gases such as an inlet port for oxygen, an inlet port for CO₂, an inlet port for nitrogen, and the like.

In some examples, the gas ports allow the headspace 20 above the wells 61 in the sample container 18 to have an oxygen concentration that ranges from 0% to 100% such that the container assembly 200 can be used for cultivating an entire range of cells from extreme anaerobes to aerobic organisms by allowing for a wide range of oxygen concentrations in the headspace 20. In some examples, the gas ports can be used to adjust the oxygen concentration in the headspace 20 to a level between 0% and 5%, 0% and 10%, or 0% and 20%.

The lid housing 8 also includes eccentric levers 7 and sealing mechanisms 9 to secure and seal the lid housing 8 to the sample container 18. The eccentric levers 7 and sealing mechanisms 9 will be discussed in more detail below.

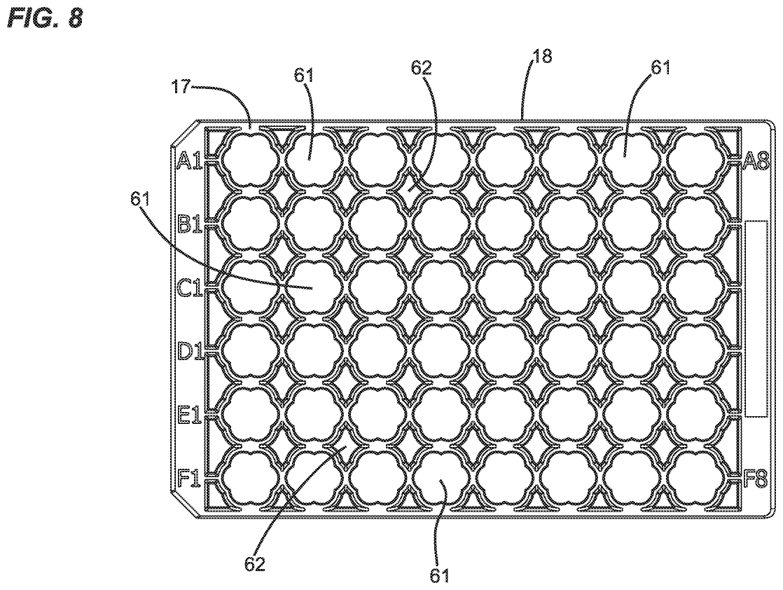

FIG. 8 is a top view of the sample container 18. As shown in FIG. 8, the sample container 18 includes a plurality of wells 61 that are arranged in rows. In the example shown in FIG. 8, the sample container 18 includes a total of 48 wells allowing a user to perform 48 parallel cultivations. Alternative sizes for the sample container 18 are possible. For example, the sample container 18 may be sized to include any of 6, 12, 24, 96, 384, or 1536 wells, and any number of wells therebetween, or any suitable number of wells.

As further shown in FIG. 8, the sample container 18 includes a sealing surface 17 that surrounds the wells 61. The sealing surface 17 includes a plurality of curved edges that are linked together, and that are semi-circularly shaped around the perimeters of the wells 61.

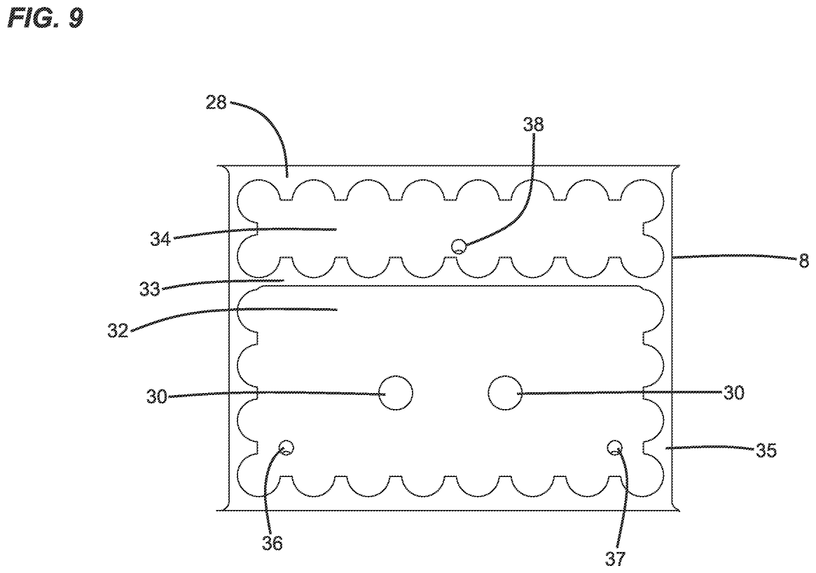

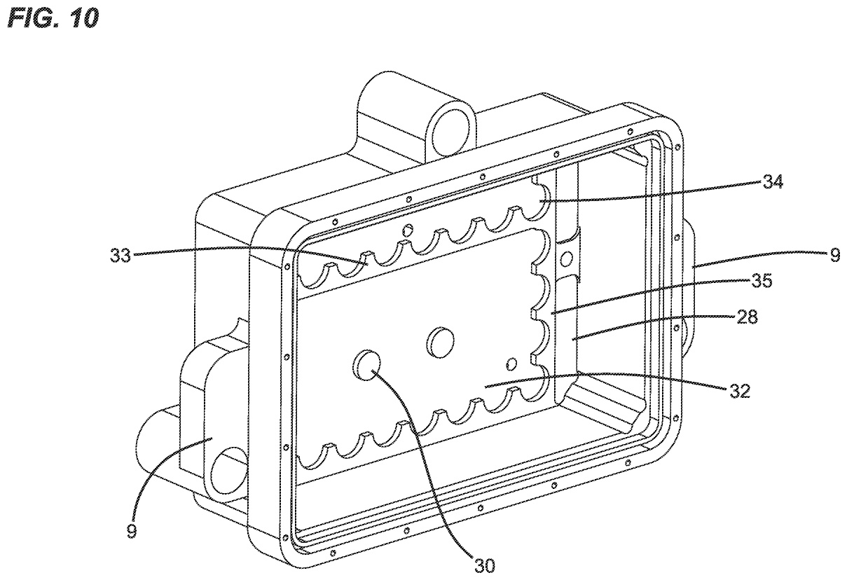

FIG. 9 is a bottom view of a first example of a lid housing 8 that is partitioned for a microplate with microfluidics. FIG. 10 is a bottom isometric view of the example of the lid housing 8 that is partitioned for a microplate with microfluidics. FIG. 11 is a bottom isometric view of the lid housing 8 that is partitioned for a microplate with microfluidics relative to the sample container 18. As shown in FIGS. 9-11, a sealing surface 35 projects from a bottom interior surface 28 of the lid housing 8. The sealing surface 35 is configured to contact and push down on the first resilient layer 13 (see also FIG. 6), which is thus caused to be compressed against the sealing surface 17 of the sample container 18, thereby forming an airtight seal between an inside perimeter of the lid housing 8 and the sample container 18. This illustrates yet another advantage of the first resilient layer 13.

The sealing surface 35 is elevated relative to first and second recessed areas 32, 34. The sealing surface 35 can act as a boundary structure between the first and second recessed areas 32, 34 and an exterior of the container assembly 200.

The sealing surface 35 conforms to the shape of the sealing surface 17 of the sample container 18 to apply an even pressure along the edges of the sealing surface 17. The sealing surface 35 is configured to surround the edges of the wells 61 with minimal intrusion inward.

A threshold amount of pressure is needed between the lid housing 8 and sample container 18 to create an air-tight seal between the sealing surfaces 17, 35. Increasing the surface area of contact between the sealing surfaces 17, 35 increases the threshold amount of pressure needed to create the air-tight seal, which can compromise the structural integrity of the sample container 18. The shape of the sealing surfaces 17, 35 reduces the contact area to an optimal region surrounding the wells 61 that minimizes the threshold amount of pressure needed to form an air-tight seal between the lid housing 8 and sample container 18.

In some examples, the sample container 18 is a microfluidic sample container. In such examples, the rows A and B of the sample container 18 (see FIG. 8) can serve as reservoir wells (e.g., wells containing media, reagents, nutrients, pH regulation liquids, or any other suitable liquids) that can feed into the other wells having cells for cultivation that are fed via microfluidic pumping processes. While an atmosphere with specific properties is to be produced in the wells 61 that are used for cultivation (referenced herein as “cultivation wells”), pressure may need to be applied to the reservoir wells so that the pumping process can be carried out. That is, a pressuring gas (e.g., nitrogen) may be introduced into the space above the reservoir wells to increase pressure and thereby cause fluid from the reservoir wells to be conveyed into the cultivation wells via microfluidic channels, as will be described further below. In order to maintain desired pressures and gas concentrations in the headspace above the cultivation wells, these regions of the sample container 18 have to be separated. Thus, a partition 33 is used to separate the first and second recessed areas 32, 34, as shown in FIGS. 9-11.

As shown in FIGS. 9-11, the partition 33 is on the bottom interior surface 28 of the lid housing 8 to create separate sections of the wells 61 that are sealed off from each other. In some examples, the sealing surface 35 and the partition 33 are continuous with one another.

As an illustrative example, the partition 33 can define the first recessed area 32 which is designated for the cultivation wells (for clarity, the first recessed area 32 is what defines the headspace 20 above the cultivation wells), and further defines the second recessed area 34 which is designated for reservoir wells.

While two separate recessed areas are shown in this example, the lid housing 8 may include additional partitions to further subdivide the wells 61. For example, a first set of wells for cultivations that need a first concentration of a gas such as oxygen may be subdivided by another partition from a second set of wells for cultivations that need a second concentration of the gas (e.g., oxygen) that is different from the first concentration.

The sealing surface 35 and partition 33 are made from a rigid material that can be pressed against the first resilient layer 13 to compress the first resilient layer 13 and thereby provide the desired level of sealing. In some examples, the sealing surface 35 and partition 33 are made from a rigid polymer material such as polyether ether ketone (PEEK).

As shown in the example provided in FIG. 9, the lid housing 8 includes two openings 36, 37 that are each respectively connected to a gas port 11, 12 shown in FIGS. 4 and 5. One of these openings is an inlet for feeding gas, the other opening is an outlet for exhausting gas. The openings 36, 37 are provided in the first recessed area 32, and can thus be used for feeding gas with a controlled concentration of air, oxygen, nitrogen, or CO2 to the cultivation wells in the sample container 18. In some examples, this gas may be humidified to a desired level. A third opening 38 is provided in the second recessed area 34, and can be connected to a third gas port 25 on the lid housing 8 (see FIG. 23) for feeding a pressurizing gas to the reservoir wells in the sample container 18 so as to pressurize the headspace above the reservoir wells and thus cause liquid from the reservoir wells to move into the cultivation wells via microfluidic channels. In this example, the lid assembly 8 is configured for microfluidic applications.

The lid housing 8 environmentally seals the sample container 18. The mixed gases that form a desired atmosphere for the cell cultivations are guided under the lid housing 8 to pass over the wells 61. In cases where the sample container 18 includes reservoir wells for feeding cultivation wells in the sample container 18, the reservoir wells can be sealed off from the cultivation wells using the partition 33 of the lid housing 8.

The partition 33 allows for a pressure applied on top of the reservoir wells to be different from a pressure applied on top of the wells that are used to cultivate the microbial cultures. The partition 33 also allows for preventing mixing of the gases over the reservoir wells with components in the cultivation wells, and thus allows for gas in the headspace above the cultivation wells to be regulated.

Still referring to FIGS. 9-11, the lid housing 8 can include one or more posts 30 that push down on the first resilient layer 13 covering the sample container 18 so as to ensure that the first resilient layer 13 does not deform beyond a prescribed limit during cultivation. For example, the first resilient layer 13 can be made of a material that may be susceptible to expanding and thus deforming (either temporarily or permanently) in an outward direction when it is exposed to heat and gases above a certain threshold. The posts 30 project from bottom interior surface 28 in the first recessed area 32 which covers the wells 61 that are configured for cell cultivation. The posts 30 extend toward the wells 61 to help prevent the first resilient layer 13 from deforming and pushing upwards (e.g., beyond a threshold amount) due to the heat, gases, or other forces that emit from the system as a whole including the wells 61 during cultivation. In some examples, the posts 30 do not extend as far as the partition 33 or the sealing surface 35, and thus do not touch or push against the resilient layer 13 when the seal is formed. Rather, the posts 30 may only touch when the resilient layer 13 deforms more than a threshold amount.

While FIGS. 9-11 show two posts on the bottom interior surface 28 of the lid housing 8, in alternative examples the lid housing 8 may include only one post, or may include more than two posts. Thus, the arrangement of the posts 30 that is shown in FIGS. 9 and 10 is provided as an illustrative example, and the lid housing 8 is not limited to this particular arrangement.

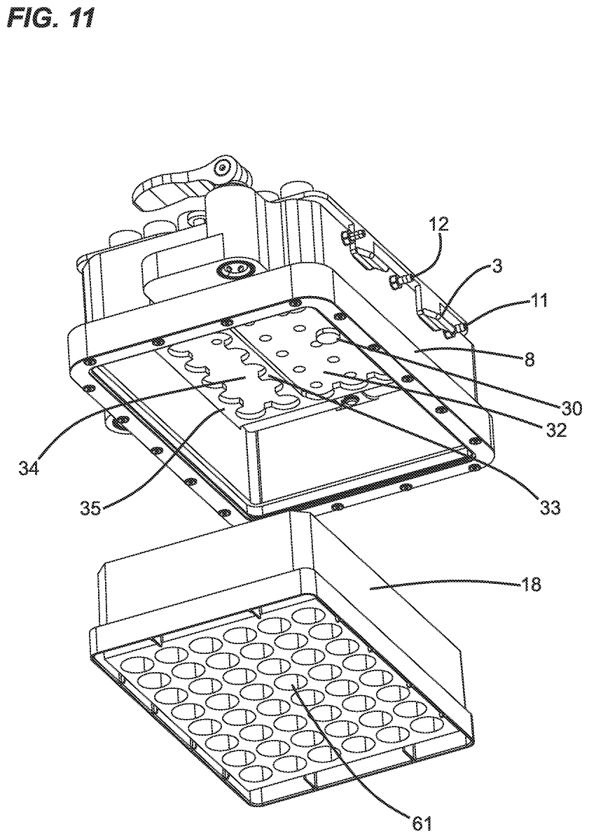

FIG. 12 is a bottom view of another example of the lid housing 8. In this example, the bottom interior surface of the lid housing 8 does not include the partition 33 shown in FIGS. 9-11, such that there is only a single recessed area 42 on the bottom interior surface of the lid housing 8. A sealing surface 45 surrounds the recessed area 42. The sealing surface 45 is substantially similar or the same as the sealing surface 35. In this example, the bottom interior surface of the lid housing 8 includes four posts 40 that project from the recessed area 42.

In the example illustrated in FIG. 12, the lid housing 8 includes two openings 36, 37 that are connected to the gas ports 11, 12 shown in FIGS. 4 and 5. The openings 36, 37 each correspond to one of an inlet gas port and an outlet gas port (e.g., gas ports 11, 12) and are provided in the single recessed area 42. The openings 36, 37 can be used for feeding gas with a controlled concentration of air, oxygen, nitrogen, or CO₂ to the cultivation wells and exhausting the gas. In this example, the lid assembly 8 is configured for non-microfluidic applications.

FIG. 13 is a cross-sectional view of the container assembly 200 showing a pipette tip 71 inserted through a guide element 2 of the lid housing 8. In this example, a pipetting robot 70 controls the movement of the pipette tip 71. FIG. 14 is a cross-sectional view of the container assembly 200 after the pipette tip 71 has been removed from the container assembly 200.

Although the pipetting robot 70 is described for controlling the movement of the pipette tip 71, the disclosure herein contemplates that sampling and introducing fluids into the wells 61 of the sample container 18 may also be performed manually. For example, a user may manually insert one or more pipette tips 71 into one or more wells 61 for sampling a fluid from the one or more wells 61 or introducing a fluid into the one or more wells 61.

As shown in FIGS. 13 and 14, the guide elements 2 each define a hollow interior portion 60 that can help guide the pipette tips 71 toward a specific location in the sample container 18 (e.g., a well 61). In some examples, the hollow interior portions 60 have a conical or frustoconical shape to help guide the pipette tip 71 through the various layers and components of the container assembly 200. This is especially advantageous when pipette tip 71 is inserted and removed during agitation or shaking of the container assembly 200 by the microbioreactor 100 when inside the cultivation chamber 104 during cultivation and/or fermentation.

During the orbital shaking motion of the container assembly 200, the pipette tip 71 may be inserted into a guide element 2 above a desired well. As soon as the diameter of the hollow interior portion 60 becomes smaller than an agitation (e.g., shaking) diameter inside the cultivation chamber 104, there is direct contact between the pipette tip 71 and the guide element 2. Due to the flexibility of the pipette tip 71 and of the connection to the pipetting robot 70, the pipette tip 71 is guided to the narrowest part of the guide element 2 and finally through the guide element 2. Thereafter, the pipette tip 71 can be pushed through the various layers and components of the container assembly 200 to reach a well 61 of the sample container 18.

For example, the pipette tip 71 can pass through an aperture 6 of the second resilient layer 4, a through-hole 23 of the lid housing 8, and an aperture 15 of the first resilient layer 13 until it reaches the sterile layer 16. The pipette tip 71 is sufficiently rigid so as to pierce a hole in the sterile layer 16, and thereafter reach a well 61 on the sample container 18. Accordingly, the guide elements 2, the through-holes 23 of the lid housing 8, and the various layers may serve to accurately position the end of the pipette tip 71 at a steady location over the sample container 18, and the size of the hole can be limited to the size of the pipette tip 71 as the sample container 18 is shaken such that the hole is not enlarged due to the shaking of the sample container 18, and accordingly, the size of the hole formed from the pipette tip 71 passing through is minimized.

Furthermore, the accurate positioning of the pipette tip 71 by the guide elements 2 ensures that the sterile layer 16 is not pierced in multiple locations over the same well during multiple insertions of the pipette tip 71. This is an advantageous feature because a single experiment may include several hundred pipetting tip insertions over a single well, and multiple holes in the sterile layer 16 over the same well may increase the risk of contamination.

The microbioreactor 100 includes an actuator system that is configured to move the container assembly 200 in an orbital fashion. Continuous shaking improves the aeration of the wells 61 and prevents sedimentation inside the wells. Thus, interrupting the shaking while pipetting into a well or out of a well is not desirable. In order to prevent the apertures 15, 6 of the first and second resilient layers 13, 4 from wearing out, the pipette tip 71 must hit the middle of the apertures 15, 6 to avoid harming the flanks of the apertures 15, 6.

To guide the pipette tip 71 to a through-hole 23 in the lid housing 8 and the middle of the apertures 15, 6 in the first and second resilient layers 13, 4, the pipette tip 71 is guided by the guide elements 2 of the guide structure 1. The guide elements 2 act like funnels for guiding the pipette tip 71 to the through-holes 23 and the middle of the apertures 15, 6 and keeping the pipette tip 71 centered in this location during agitation of the container assembly 200. After the pipette tip 71 is removed from the apertures 15, 6, the apertures close by themselves through the elastic nature of the first and second resilient layers 13, 4.

In some examples, the actuator system of the microbioreactor 100 is configured to move the container assembly 200 in an orbital fashion within a range of 600 RPM to 1000 RPM, and with an agitation diameter of 1-6 mm. In some further examples, the actuator system of the microbioreactor 100 is configured to move the container assembly 200 in an orbital fashion within a range of 600 RPM to 800 RPM, and with an agitation diameter of 1-5 mm. In some further examples, the actuator system of the microbioreactor 100 is configured to move the container assembly 200 in an orbital fashion within a range of 100 RPM to 1000 RPM, and with an agitation diameter of 1-5 mm. In some further examples, the actuator system of the microbioreactor 100 is configured to move the container assembly 200 in an orbital fashion within a range of 600 RPM to 800 RPM, and with an agitation diameter of 3 mm. In some further examples, the actuator system of the microbioreactor 100 is configured to move the container assembly 200 in an orbital fashion within a range of 100 RPM to 2000 RPM, and with an agitation diameter of 1-30 mm.

After the sterile layer 16 is pierced by the pipette tip 71, the first resilient layer 13 maintains a seal over the wells 61 of the sample container 18. For example, the first resilient layer 13 includes an aperture 15 over each of the wells 61, and the apertures 15 are “self-healing” in that they can automatically seal in on themselves when not penetrated by the pipette tip 71.

The first and second resilient layers 13, 4 can be made from any suitable resilient and compliant material. In some examples, the first and second resilient layers 13, 4 are silicone films, which provide suitable resilience and compliance. Alternatively, the first and second resilient layers 13, 4 can be made from soft polymers, or hard polymers blended with a softener.

The pipette tip 71 is precisely guided into an aperture 15 of the first resilient layer 13 by the guide elements 2 described above. Each of the apertures 15 opens due to the pressure of the pipette tip 71, such that said pipette tip 71 can pierce the sterile layer 16 and dip into a well 61 as shown in FIG. 13. After the pipetting procedure has ended, the pipette tip 71 is pulled out of the well 61. Without the pressure exerted by the pipette tip 71, the aperture 15 of the first resilient layer 13 closes on its own in a gas-tight manner and thus provides a seal over the sterile layer 16 when damaged.

FIGS. 13 and 14 show the self-healing nature of the apertures 15 of the first and second resilient layers 13, 4. In FIG. 13, the apertures 15, 6 are opened when the pipette tip 71 is inserted, and in FIG. 14, the apertures 15, 6 are closed after the pipette tip 71 is removed.

The through-holes 23 can cause gases to leak from the headspace 20 of the lid housing 8. Thus, the second resilient layer 4 is applied over the through-holes 23 of the lid housing 8. In some examples, the second resilient layer 4 is self-adhesive on at least one side.

The apertures 6 of the second resilient layer 4 are aligned above each respective through-hole 23 of the lid housing 8. The apertures 6, like the apertures 15, are self-healing such that they are configured to open when the pipette tip 71 is pushed through, and after the pipetting procedure, the apertures 6 close on their own to seal the headspace 20.

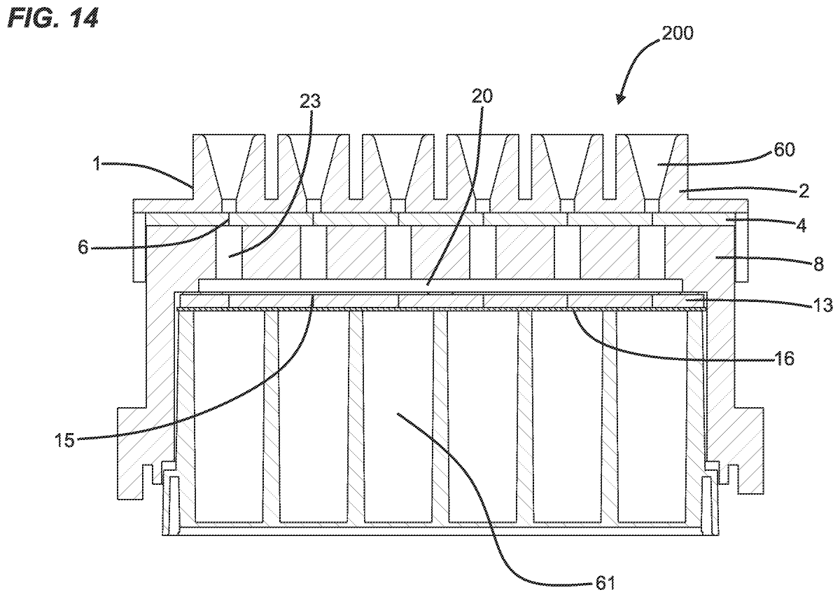

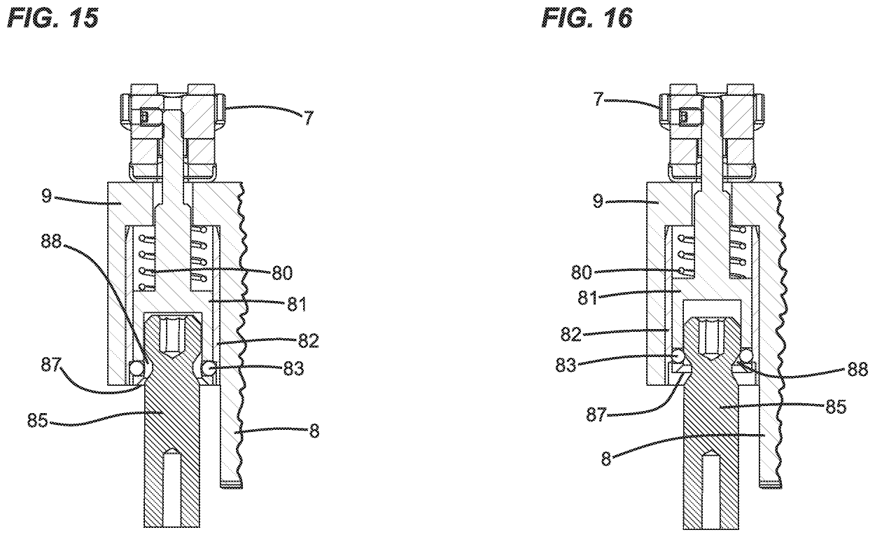

FIG. 15 is a cross-sectional view showing a sealing mechanism 9 in an opened condition. FIG. 16 is a cross-sectional view showing the sealing mechanism 9 in a closed condition. The sealing mechanism 9 is configured to press the lid housing 8 onto the sample container 18 with a sufficient amount of pressure to create an air-tight seal between the lid housing 8 and the sample container 18, without damaging to the sample container 18.

The sealing mechanism 9 includes a press sleeve 82 that is mounted inside the sealing mechanism 9 by a press-fit connection. The press sleeve 82 is vertically aligned with a ball sleeve 81 inside the sealing mechanism 9. The ball sleeve 81 terminates in a shoulder 87 that engages radially guided balls 83. Based on the vertical position of the shoulder 87, the radially guided balls 83 open or close the seal between the lid housing 8 and a base post 85 (see FIG. 19).

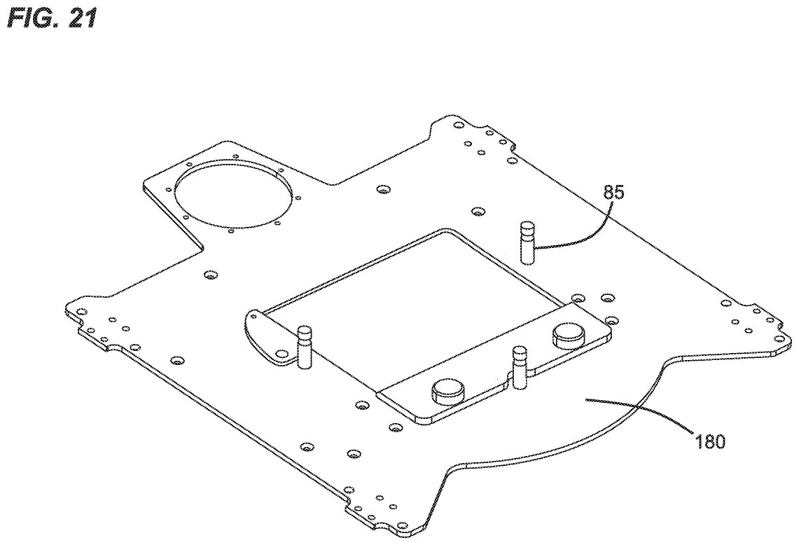

The ball sleeve 81 is biased by a spring 80 to be pushed down in the opened position. When the eccentric lever 7 is actuated, the shoulder 87 is pulled up. The diameter of the press sleeve 82 is decreased when the radially guided balls 83 are pulled upward which causes the radially guided balls 83 to be displaced in a radial direction toward a groove 88 of the base post 85 of an orbital shaking platform 180 (see FIGS. 19 and 21). This produces a form fit between the lid housing 8 and the base post 85, and thereby attaches the lid housing 8 to the base post 85.

Additionally, the sealing mechanisms 9 causes the lid housing 8 to press onto the sample container 18 to create a sealed gas atmosphere above the sample container 18 when the sample container 18 is held inside the cultivation chamber 104 of the microbioreactor 100. Due to the generated torque, the eccentric lever 7 is self-locking in the closed position.

When the eccentric lever 7 is operated to move from the closed position to the opened position, the radially guided balls 83 are pushed downwards where the diameter of the press sleeve 82 is increased, such that the radially guided balls 83 expand in a radial direction away from the groove 88 to release the lid housing 8 from the base post 85. Thus, the sealing mechanism 9 provides an easy way to attach and detach the lid housing 8 from the base post 85, as well as to press the lid housing 8 onto the sample container 18, and to release it therefrom.

When the lid housing 8 is pressed onto the sample container 18, a seal is created between the lid housing 8 and sample container 18 by the geometrically unique elevations of the sealing surface 35 and partition 33 inside the lid housing 8, and the sealing surface 17 on the sample container 18. The shape of the sealing surfaces 17, 35 reduces the pressure from the sealing mechanism 9 and eccentric levers 7 needed for pressing the lid housing 8 onto the sample container 18, allowing the lid housing 8 to seal the sample container 18 in a gas-tight manner without damaging the sample container 18.

FIG. 17 is a cross-sectional view of the lid housing 8 and sample container 18. The lid housing 8 can further include a seal 90 that engages and compresses itself against an exterior sidewall of the sample container 18 to provide another seal between the lid housing 8 and sample container 18. The seal 90 can be an anaerobic seal that blocks gases from entering or escaping from the sample container 18 before the sample container is inserted into the cultivation chamber 104 of the microbioreactor 100. For example, there can be a need in some instances for an oxygen-free atmosphere inside the sample container 18, such that the container assembly 200 is configured for use as an anaerobic chamber. The seal 90 can prevent oxygen from entering the wells 61 of the sample container 18.

In view of the foregoing, a sample can be added to the sample container 18 in an anaerobic tent, and the sample container 18 can be sealed with the lid housing 8 before the container assembly 200 is taken to the microbioreactor 100, which may be located outside the anaerobic tent, for cultivation and/or further work. This is advantageous in that it allows users to work with the cultures freely without special equipment, and further advantageous in that the microbioreactor does not need to be positioned within the anaerobic tent and can thereby be more accessible. Accordingly, the anaerobic seal between the sample container 18 and lid housing 8 allows for easy transport of the container assembly 200 in an open-air environment.

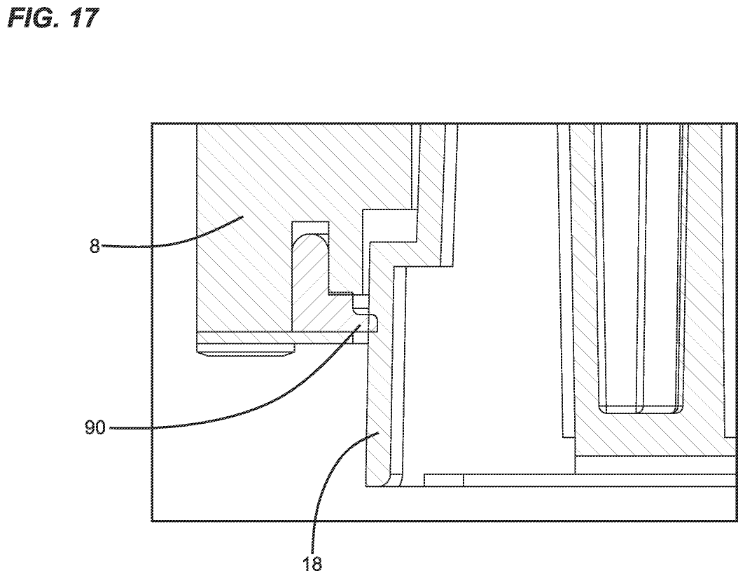

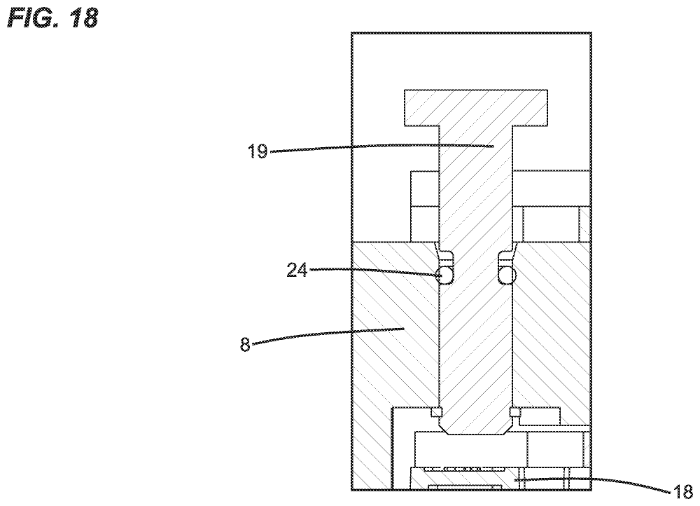

FIG. 18 is a cross-sectional view showing a release pin 19 of the container assembly 200. As shown in FIG. 18, the release pins 19 are each sealed by an O-ring 24, and can be used to release the lid housing 8 from the sample container 18. For example, the sample container 18 can be released from the lid housing 8 by holding the lid housing 8 and exerting pressure on the release pins 19 to push or eject the sample container 18 out of the lid housing 8.

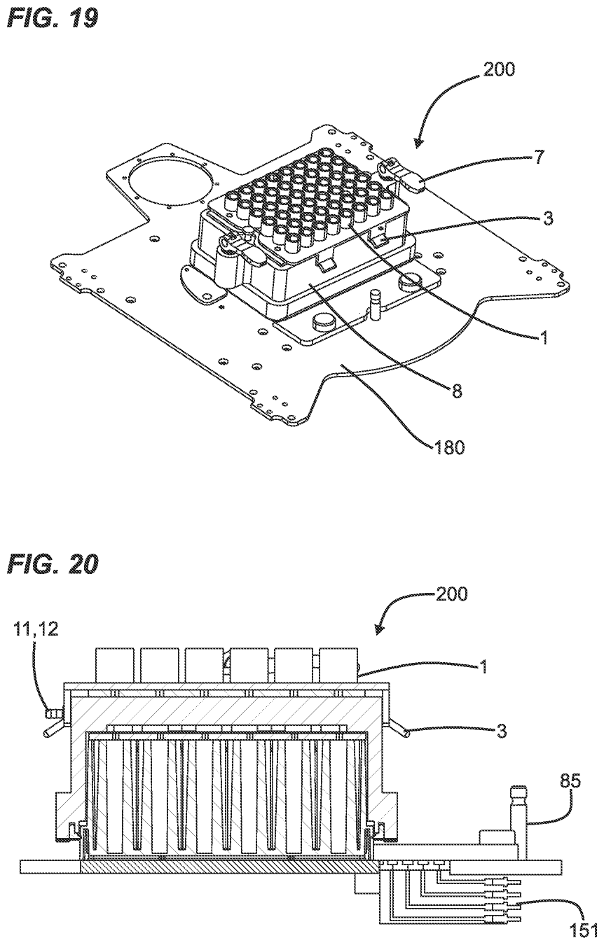

FIGS. 19-21 show an example of the container assembly 200 that is configured for non-microfluidic applications. In this example, the container assembly 200 has two eccentric levers 7. FIG. 19 shows the container assembly 200 mounted to an orbital shaking platform 180, FIG. 20 shows a cross-sectional view of the container assembly 200 mounted to the orbital shaking platform 180 without connection to microfluidic gas channels 151, and FIG. 21 shows the orbital shaking platform 180 when the container assembly 200 is not mounted thereto.

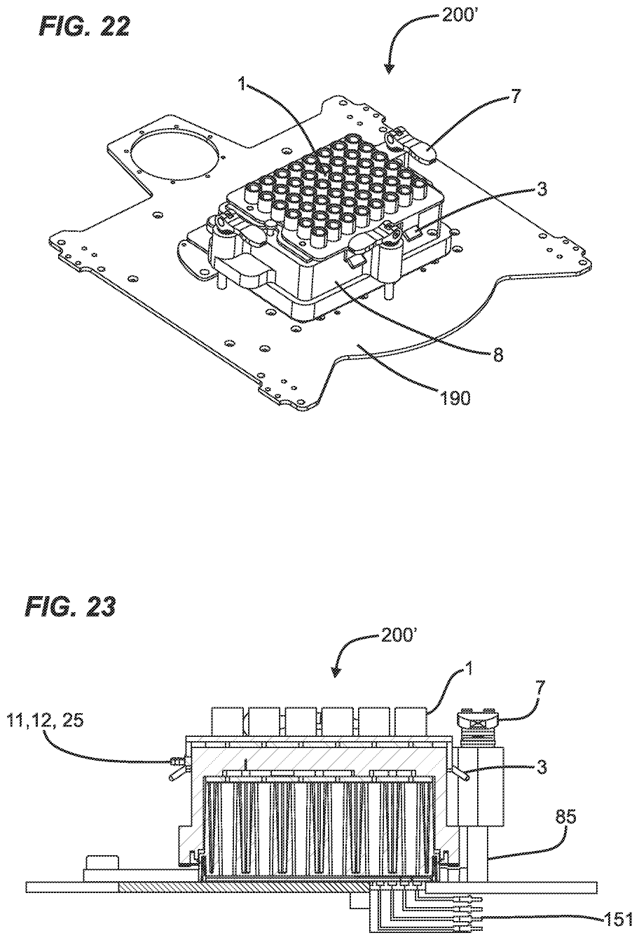

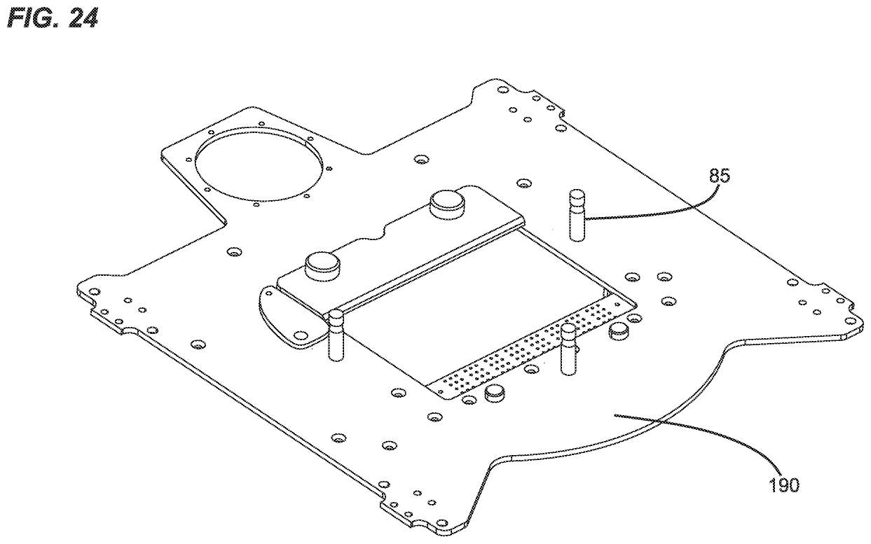

FIGS. 22-24 show views of an example of the container assembly 200′ that includes three gas ports 11, 12, 25 for microfluidic applications. FIG. 22 shows the container assembly 200′ mounted to an orbital shaking platform 190, FIG. 23 shows a cross-sectional view of the container assembly 200′ mounted to the orbital shaking platform 190 with connections to the microfluidic gas channels 151, and FIG. 24 shows the orbital shaking platform 190 when the container assembly 200′ is not mounted thereto. In this example, three eccentric levers 7 are provided on the container assembly 200′ because the levers are offset on the sides. By providing the third lever, pressure can be uniformly distributed around the entire assembly. Otherwise, the offset levers would not distribute pressure uniformly.

The microfluidic gas channels 151 are used for operating microfluidic valves that control the flow of reagents in the reservoir wells that are fed into the cultivation wells. The microfluidic gas channels 151 are used to pressurize the microfluidic valves such that the microfluidic valves are closed when pressure is applied to them and are opened when no pressure is applied. Through a specific order in which the microfluidic valves are opened and closed, a defined volume of reagents can be fed into the cultivation wells. In some examples, there are 96 microfluidic gas channels 151 which control each of microfluidic valve individually. This technology is described in more detail in U.S. Pat. No. 8,932,544, titled Microreactor Array, Device Comprising a Microreactor Array, and Method for Using a Microreactor Array, issued on Jan. 13, 2015, the entirety of which is hereby incorporated by reference.

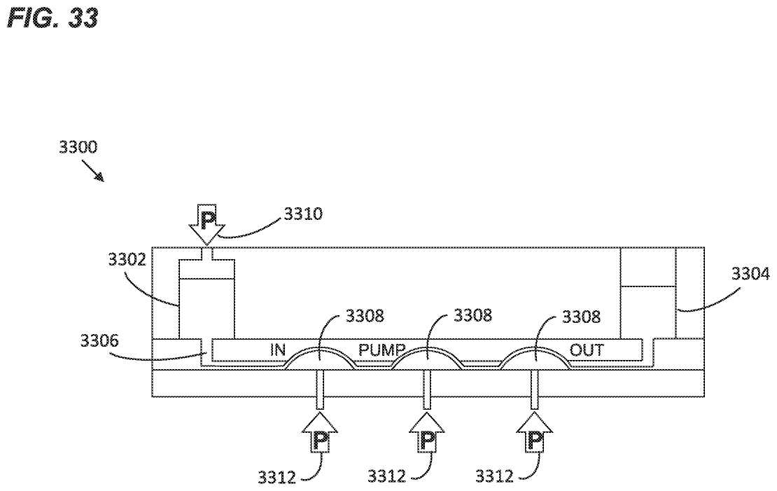

FIG. 33 illustrates a microfluidic valve configuration 3300 where a pressurizing gas 3310 pressurizes a headspace above a reservoir well 3302 causing liquid from the reservoir well 3302 to move down into a fluid duct 3306. A controlled sequence of feeding pressuring gas 3312 from the microfluidic gas channels 151 causes microfluidic valves 3308 along the fluid duct 3306 to open and close. The opening and closing of the microfluidic valves 3308 causes the liquid to move across the fluid duct 3306 until it reach the cultivation well 3304.

Microbial cultures require a gas atmosphere to grow. For most cells, oxygen is a critical component of the atmosphere. However, pure oxygen can be toxic to organisms such that it is often diluted with nitrogen to create an atmosphere like air with varying concentrations of oxygen. CO₂ from the atmosphere can be used to adjust the pH or as a carbon source for phototrophic organisms conducting photosynthesis. The container assembly 200 can be used to provide an atmosphere for the wells 61 having a mixture of air, oxygen, nitrogen, and CO₂.

The gases listed above can be mixed during an experiment. In some examples, two of the gases can be mixed. For example, to increase the oxygen concentration of the atmosphere, air can be mixed with oxygen. To decrease the oxygen concentration of the atmosphere, air can be mixed with nitrogen. To increase the CO₂ concentration of the atmosphere, air can be mixed with CO₂. In some examples, only two gases are mixed at a time. In alternative examples, more than two gases can be mixed.

In the above examples, the mixture is created by two or more valves controlled with a pulse-width-modulation (PWM) signal to set a ratio between the time each valve is opened or closed. The gas is fed through a gas inlet port (e.g., one of the gas ports 11, 12 in the example figures). The longer the time the valve is opened, higher the concentration of the gas that can pass the valve. After the gases are mixed, a sensor can measure the oxygen or CO₂ levels in the atmosphere. The control feedback is a controller that can adjust the PWM signal accordingly to reach the predefined values.

To avoid the loss of liquid in the sample container 18, the gas introduced into the container assembly 200 through the lid housing 8 can be saturated with humidity. This prevents evaporation of the medium in the wells 61 dedicated for cell cultivations. Therefore, the gas stream that is ultimately fed through one or more of the gas ports 11, 12, 25 may be led through a reservoir filled with water (or some other suitable liquid for humidifying the gas stream) at a suitable point along its flow (e.g., at some point between a source of the gas and the inlet gas port) so as to humidify the gas stream. For example, the gas stream that is fed into a gas inlet port (e.g., one of the gas ports 11, 12) may originate at one or more gas sources (e.g., gas canisters), can be mixed with another gas, and can then pass through a water reservoir, and ultimately be fed into the gas inlet port. In some examples, a tube may guide the gas stream to the bottom of the reservoir so that it has to pass through the water. In order to maximize the absorption of water in the reservoir, it is heated by a heat pad to a temperature set well above room temperature. In some examples, the additional gas port 25 (which feeds pressurizing gas into the space above the reservoir wells) may be humidified by a similar process. In other examples, the additional gas port 25 may not be humidified.

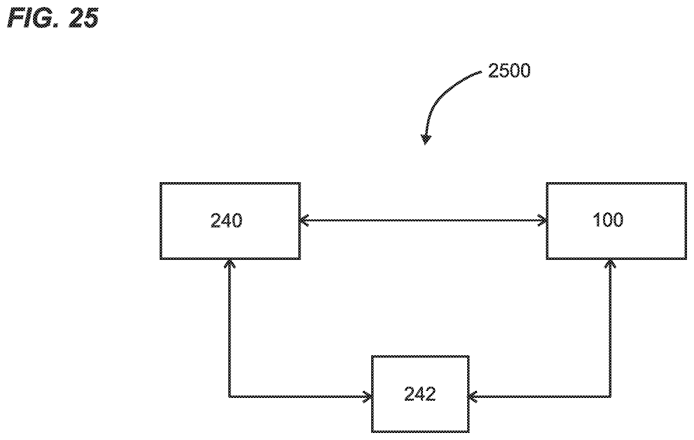

FIG. 25 schematically shows an example of a computer control system 2500 of the microbioreactor 100. As shown in FIG. 25, the computer control system 2500 includes a computer controller 240 that is operatively coupled to control the operations of the microbioreactor 100, as described above. The computer controller 240 is therefore operatively coupled to gas supplies, gas valves, sensors, actuators, and pipetting robot (collectively 242) in order to carry out the above-described functionalities. The computer controller 240 also comprises a computer storage medium that stores, in a tangible and non-transitory manner, a computer program product, that when executed by the computer controller 240, causes the computer controller 240 to carry out the above-mentioned functionalities.

Another embodiment includes at least one computer-readable medium storing data instructions that, when executed by at least one processing device (such as a processor of the computer controller 240), cause the at least one processing device to carry out one or more of the above-mentioned functionalities. For example, one embodiment includes at least one computer-readable medium storing data instructions that, when executed by at least one processing device, cause the at least one processing device to: sense measurement parameters associated with a sample container assembly having a gassing lid: process the sensed measurement parameters: and control a gas supply of at least one gas to the gassing lid based upon the processed measurement parameters.

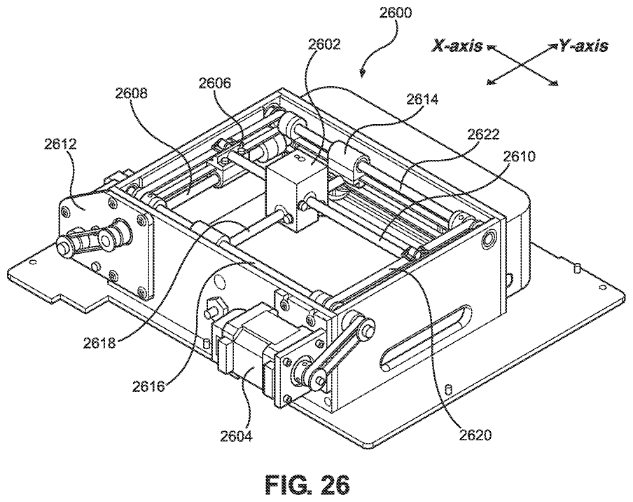

FIG. 26 is an isometric view of an example of a mechanical system 2600 of the microbioreactor 100. The mechanical system 2600 may be used by the microbioreactor 100 to position an optical sensor 2602 under the container assembly 200 when the container assembly 200 is held inside the cultivation chamber 104 of the microbioreactor 100.

The optical sensor 2602 is moved under each well 61 of the sample container 18, or under a subset of the wells 61 of sample container 18, to obtain measurements of the cell cultures in each well. The movement of the optical sensor 2602 is controlled by the mechanical system 2600 along two perpendicular axes (e.g., X and Y axes). Moving the optical sensor 2602 along the X and Y axes allows the optical sensor 2602 to be positioned under each well 61 of the sample container 18 to illuminate each well 61 with light, and to receive the scattered light that is returned back from the well 61 to obtain a measurement of one or more parameters such as biomass, pH, dissolved oxygen (DO), and fluorescence.

A first motor 2604 powers actuators 2606 to slide along shafts 2608 parallel to the Y-axis to control the position of the optical sensor 2602 along the Y-axis. The actuators 2606 carry a shaft 2610 that is parallel to the X-axis, and that is connected to the optical sensor 2602. The actuators 2606 are configured to move the optical sensor 2602 along a shaft 2618 to thereby control the position of the optical sensor 2602 along the Y-axis.

A second motor 2612 powers actuators 2614 to slide along shafts 2616 parallel to the X-axis to control the position of the optical sensor 2602 along the X-axis. The actuators 2614 are connected to the optical sensor 2602 via the shaft 2618 to move the optical sensor 2602 along the shaft 2610 to thereby control the position of the optical sensor 2602 along the X-axis.

In some examples, the first and second motors 2604, 2612 are step motors. In the example shown in FIG. 26, the first and second motors 2604, 2612 pull belts 2620, 2622 respectively to control the movement of the actuators 2606, 2614 along the perpendicular axes. Alternative examples are contemplated for moving the optical sensor 2602 along the perpendicular axes for positioning the optical sensor 2602 under each well 61.

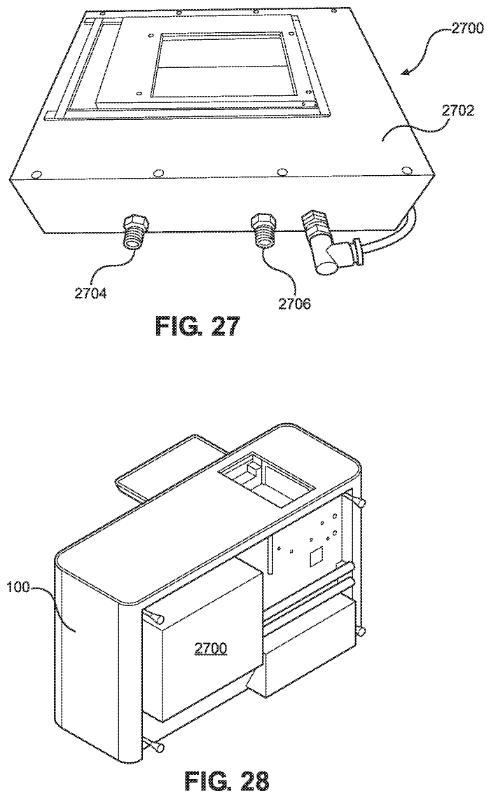

FIG. 27 is an isometric view of a light-emitting diode array module (LAM) 2700 that can be used to illuminate the cultivation chamber 104 of the microbioreactor 100. The LAM 2700 can be an add-on module. The illumination from the LAM 2700 is similar to bright sunlight. The spectral composition of the light can be varied. The LAM 2700 allows for the high-throughput cultivation of phototrophic microorganisms within the microbioreactor 100.

The LAM 2700 includes a housing 2702. In some examples, the housing 2702 is made from aluminum. In some examples, the housing 2702 measures approximately 35 cm×26 cm×9.75 cm. Alternative materials and size measurements for the housing 2702 are possible.

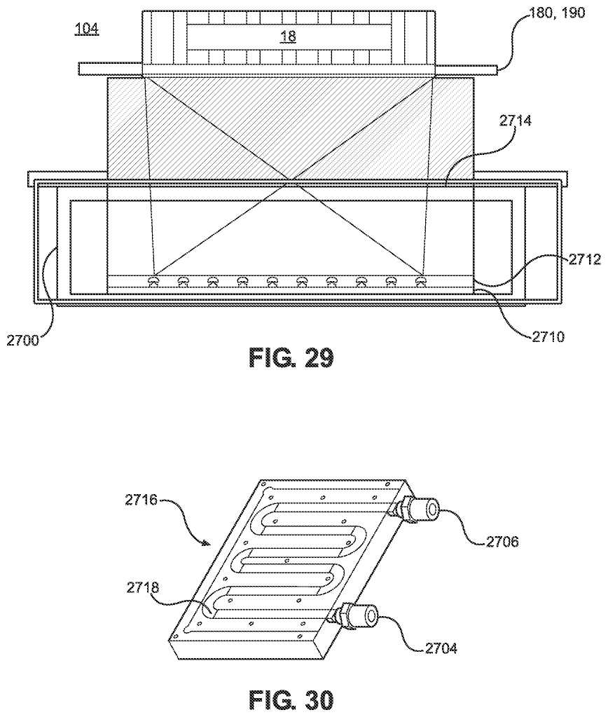

FIG. 28 is a bottom isometric view of the LAM 2700 mounted underneath the microbioreactor 100. FIG. 29 is a schematic diagram of the LAM 2700. Referring now to FIGS. 28 and 29, the LAM 2700 is configured to homogeneously illuminate the bottom of the sample container 18 (e.g., a microplate or microtiter plate) placed on an orbital shaking platform 180, 190 (e.g., a shaker) inside the cultivation chamber 104 of the microbioreactor 100.

The LAM 2700 includes an array of light-emitting diodes (LEDs) 2710 that emits the light to the illuminate the cultivation chamber 104, and can include a lens 2712 to focus the light and a transparent quartz plate 2714 that allows the light to pass through, and that protects the internal components of the LAM 2700 including the arrays of LEDs 2710 and lens 2712.

The LED 2710 can generate a considerable amount of heat over time. Thus, the LAM 2700 includes a cooling plate 2716 (see FIG. 30) that can be used to cool down the LAM 2700 and/or the cultivation chamber 104 of the microbioreactor 100.

FIG. 30 is an isometric view of the cooling plate 2716. Referring now to FIGS. 27 and 30, the cooling plate 2716 includes an inlet 2704 that receives a liquid coolant (e.g., water) that runs through a coil 2718 to cool down the LAM 2700 before exiting through an outlet 2706. The coil 2718 can have a serpentine shape to increase the surface area of the coil 2718 and thereby increase the cooling effect of the liquid coolant that runs through it.

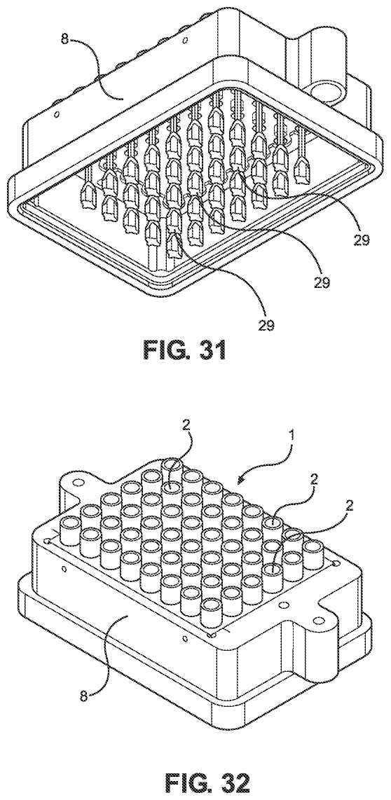

FIG. 31 is a bottom isometric view of an example of the lid housing 8 that is adapted to cool the sample container 18 (e.g., a microplate or microtiter plate). FIG. 32 is a top isometric view of the lid housing 8. In this example, the lid housing 8 (e.g., gassing lid) includes cooling pins 29 that connect to the guide elements 2 on the one end, and that extend downward into gaps 62 between the wells 61 of the sample container 18 (see FIG. 8). The cooling pins 29 and guide elements 2 can be made of conductive materials and can thus act as a heat sink for taking heat away from the sample container 18 and diffusing it to ambient air outside of the container assembly 200. In some examples, to dissipate the heat more quickly and to improve the thermal contact between the cooling pins 29 and sample container 18, the gaps 62 between the wells 61 of the sample container 18 can be filled with a liquid, such as demineralized water.

Thus, the cooling pins 29 can create a heat exchange between the sample container 18 and well-tempered air in the upper portion of the cultivation chamber 104, while at the same time serves as a guide for the pipette tip 71. The cooling pins 29 can maintain the temperature inside the sample container 18 at an acceptable level, and that is uniformly distributed within the sample container 18. Also, the cooling pins 29 can help to maintain the temperature inside the upper and lower portions of the cultivation chamber 104 at an acceptable level.

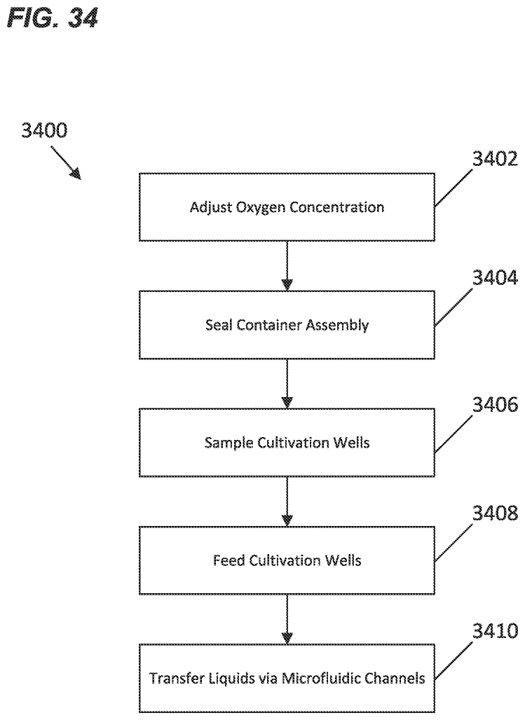

FIG. 34 illustrates an example of a method 3400 of anaerobic cultivation that can be performed using the container assembly 200. The method 3400 includes an operation 3402 of adjusting oxygen concentration in the headspace above the cultivation wells to a predetermined oxygen range that is below a threshold amount (e.g., between 0%-5%, 0%-10%, any range in between) while in the anaerobic environment. Next, the method 3400 includes an operation 3404 of sealing the container assembly 200. In accordance with the examples described above, the container assembly 200 can be sealed using the eccentric levers 7.

The method 3400 next includes an operation 3406 of sampling from cultivation wells through the various layers and components of the container assembly 200. For example, a pipette tip 71 can be inserted through a guide element 2, through an aperture 6 of the second resilient layer 4, through a through-hole 23 of the lid housing 8, and through an aperture 15 of the first resilient layer 13, such that the pipette tip 71 can pierce the sterile layer 16, and obtain a sample from a cultivation well of the sample container 18. In some examples, the pipette tip 71 can be operated by the pipetting robot 70. Alternatively, the pipette tip 71 can be operated by hand. Operation 3406 can be performed while the container assembly 200 maintains the anaerobic atmosphere in the headspace above the cultivation well, as set by operation 3402.

In some examples, the method 3400 can include an operation 3408 of adding reagents, media, or pH to the cultivation wells through the various layers and components of the container assembly 200. For example, a pipette tip 71 can be inserted in accordance with the description provided above with respect to operation 3406 for adding reagents, media, or pH to the cultivation wells. Operation 3406 can be performed while the container assembly 200 maintains the anaerobic atmosphere in the headspace above the cultivation well.

In some examples, the method 3400 can include an operation 3410 of feeding liquids such as reagents, media, or pH adjustment solution via integrated microfluidics from reservoir wells to cultivation wells to feed the cultivation wells or adjust the pH in the cultivation wells. Operation 3410 can be performed in examples where the sample container 18 is integrated with microfluidics such as on the orbital shaking platform 180, 190.

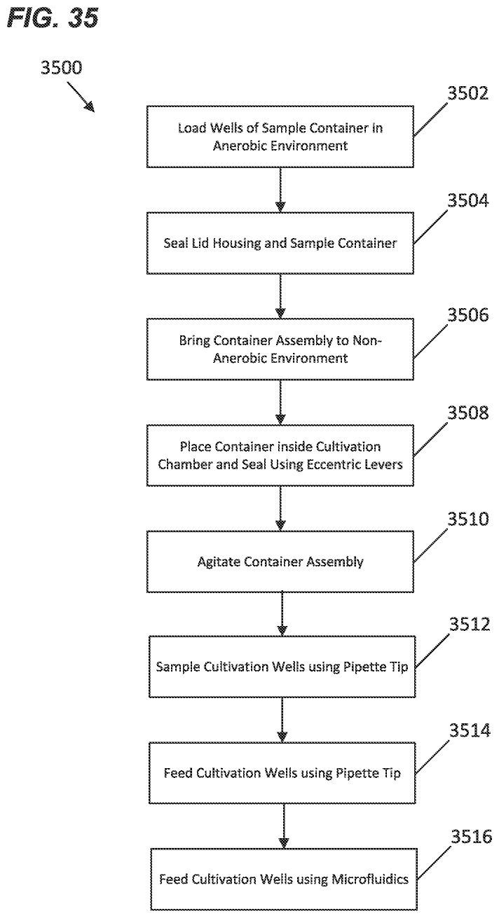

FIG. 35 illustrates another example of a method 3500 of anaerobic cultivation that can be performed using the container assembly 200. The method 3500 includes an operation 3502 of loading the wells 61 of the sample container 18 (e.g., microtiter plate or microplate) with cells and media in an anaerobic environment. In some examples, the anaerobic environment is an anaerobic tent that has a very low oxygen concentration.

Next, the method 3500 includes an operation 3504 of sealing the lid housing 8 onto the sample container 18 using the seal 90. As described above, the seal 90 prevents oxygen from entering the wells 61 of the sample container 18.

Next, the method 3500 includes an operation 3506 of bringing the container assembly 200 outside the anaerobic environment to a non-anaerobic environment. In some examples, the non-anaerobic environment refers to an environment that is outside of the anaerobic tent such as the normal environment of a lab. In some examples, the non-anaerobic environment is where the microbioreactor 100 is located.

Next, the method 3500 includes an operation 3508 of placing the container assembly 200 into the cultivation chamber 104 of the microbioreactor 100 and sealing the container assembly using the sealing mechanism 9 with the eccentric levers 7.

Next, the method 3500 includes an operation 3510 of continuously or semi-continuously agitating the container assembly 200 inside the cultivation chamber 104. For example, the container assembly 200 can be agitated by motion of the orbital shaking platform 180, 190 on which the container assembly 200 is seated or attached.

Next, the method 3500 can include an operation 3512 of sampling the cultivation wells inside the container assembly 200 with a pipette tip 71 such as by removing some of the liquid in the cultivation wells. Operation 3512 can be performed while the container assembly 200 is being agitated (see operation 3510). In some examples, the pipette tip 71 is operated by the pipetting robot 70. Alternatively, the pipette tip 71 can be operated by hand, either singly or using a multi-pipette tool. Operation 3512 can be similar to operation 3406 described above with respect to the method 3400.

Next, the method 3500 can include an operation 3514 of feeding the cultivation wells with reagents, nutrients, or media with the pipette tip 71. Operation 3514 can be performed while the container assembly 200 is being agitated (see operation 3510). In some examples, the pipette tip 71 is operated by the pipetting robot 70. Alternatively, the pipette tip 71 can be operated by hand. Operation 3514 can be similar to operation 3408 described above.

Next, the method 3500 can include an operation 3516 of feeding the cultivation wells with reagents, nutrients, or media via integrated microfluidics (e.g., the described pneumatic valve system at the bottom of the sample container 18). Operation 3516 can be similar to operation 3410 described above with respect to the method 3400.

Probiotics are living bacteria that have health-promoting benefits and bio-functional effects on the human organism. They are commonly used to increase the number of desirable bacteria in the intestine and to regenerate the intestinal flora, for example after antibiotic treatments. That is one reason why the market for probiotics or probiotic nutritional supplements has greatly increased in value. The research field of the human intestinal microbiome and its health-promoting benefits is particularly important for the nutrition industry. Therefore, scientific research on anaerobic or microaerophilic cultivation techniques, such as the cultivation of probiotics under microbiome-like conditions, is essential. Probiotics include a whole range of anaerobic bacteria such as Lactobacillus or Bifidobacterium. Among the various probiotic bacteria, Bifidobacterium spp. is one of the most widely used and studied probiotic bacterium species. They are classified as strict anaerobes due to the incapability of oxygen respiration and growth under aerobic cultivation conditions, and they are a major member of the dominant human gut microbiota. They play a significant role in controlling the pH through the release of lactic and acetic acids, which restrict the growth of many potential pathogenic bacteria. In the intestinal tract of breast-fed infants, Bifidobacterium is the predominant cell species. It accounts for more than 80% of microorganisms in the intestine. There are more than 200 known species of Lactobacillus, the largest and most diverse genus within the lactic acid bacteria which that is generally recognized as safe (GRAS) by the US Food and Drug Authority Administration (FDA). Lactobacillus spp. have been deployed and studied extensively as fermentation starter cultures for dairy products or probiotics due to their applied health potential.

In this application, anaerobic cultivation experiments can be performed using the container assembly 200 which includes the sample container 18 in combination with the gassing lid. The container assembly 200 is a bench-top device for high-throughput screening of microbial cultivations that enables online-monitoring of the most common cultivation parameters such as biomass, pH value, oxygen saturation of the liquid phase (DO) and fluorescence intensity of various fluorescing molecules or proteins. To achieve high throughput, cultivations are carried out in SBS/SLAS standard format microtiter plates (e.g., the sample container 18) with 48 wells each, which allows for the simultaneous run of up to 48 batches in the container assembly 200. Furthermore, the simplicity of using the gassing lid to perform anaerobic batch and fed-batch cultivations of the probiotic bacteria Lactobacillus casei, Lactobacillus plantarum, and Bifidobacterium bifidum. A main advantage of the gassing lid is that feeding and pH control can now take place simultaneously during direct nitrogen (e.g., 100% N2) gassing of the sample container 18 with adjustable flowrates between 5-50 mL/min.

### Anaerobic Cultivations of Lactobacillus Strains

All cultivations of Lactobacillus spp. (Lactobacillus casei DSM 20011 or Lactobacillus plantarum DSM 20174) took place in MRS broth at 37° C. ambient temperature and under anaerobic conditions. MRS broth was enriched with 0.5 g/L cysteine-HCl which serves as reducing agent for oxidation-reduction potential by reducing the residual molecular O₂ in the medium. All precultures were performed in a 250 mL Erlenmeyer flask. For this purpose, 20 mL of prepared MRS broth was inoculated with 1 mL cryoculture and then cultivated for at least 24 hours under anaerobic conditions. The main culture was then set to ODₛₜₐᵣₜ=1 in MRS broth. Subsequent microbioreactor cultivations were performed in a microfluidic round well plate for pH-controlled batch and fed-batch cultivations. The cultivations were conducted at 37° C., 600 rpm and enabled humidity control. The start volumes of the cultivation wells were set to 2,000 μL and the maximum volumes to 2,400 μL. Online monitoring of biomass (gain 3) and the measurement of pH (LG1) and dissolved oxygen DO (RF) were performed by the microbioreactor 100. A more detailed overview of the fed-batch cultivation conditions of L. casei are shown in table 1.

TABLE 1
Fed-batch cultivation conditions for L. casei

| | CONTENT | MICROFLUIDIC SETTINGS |
|---|---|---|
| RESERVOIR A | 500 g/L glucose | Pump volume: 0.16 μL |
| (FEED) | | Filling volume: 1,900 μL |
| | | Feed-start: >7.5 h or >10 h |
| | | Constant feed: 4 L/h |
| RESERVOIR B | 3M NaOH | Pump volume: 0.30 μL |
| (PH-CONTROL) | | Filling volume: 1,900 μL |
| | | pH-control-start: >0.5 h |
| | | PI settings: MEDIUM |
| CULTIVATION | L. casei in MRS broth | Start volume: 2000 μL |
| WELLS | | Max. volume: 2400 μL |
| | | pH-control: pH 6.0 |

### Anaerobic Cultivations of B. bifidum in the Microbioreactor

All cultivations of Bifidobacterium bifidum were performed in MRS broth at 37° C. and under anaerobic conditions. MRS broth was enriched with 0.5 g/L cysteine-HCl which serves as reducing agent for the oxidation-reduction potential by reducing the residual molecular O₂ in the medium. The preculture cultivations took place in a 250 mL Erlenmeyer flask. For this purpose, 20 mL MRS broth was inoculated with the content of one capsule and then cultivated for at least 24 h at 37° C. under anaerobic conditions. The main culture was set to ODₛₜₐᵣₜ=1.0 in MRS broth.

For the main culture in the container assembly 200, pH-controlled batch and fed-batch cultivations at 37° C., 600 rpm, enabled humidity control, online monitoring of biomass (gain 3), pH (LG1), and DO (RF) were performed. A more detailed overview of the fed-batch cultivation conditions of B. bifidum are listed in table 2.

TABLE 2
Fed-batch cultivation conditions for B. bifidum

| | CONTENT | MICROFLUIDIC SETTINGS |
|---|---|---|
| RESERVOIR A | 500 g/L glucose | Pump volume: 0.16 μL |
| (FEED) | | Filling volume: 1,900 μL |
| | | Feed-start: >5 h |
| | | Constant feed: 4 μL/h |
| RESERVOIR B | 3M NaOH | Pump volume: 0.30 μL |
| (PH-CONTROL) | | Filling volume: 1,900 μL |
| | | pH-control-start: >0.5 h |
| | | PI settings: MEDIUM |
| CULTIVATION | B. bifidum in MRS | Start volume: 2000 μL |
| WELLS | broth | Max. volume: 2400 μL |
| | | pH-control: pH 6.0 |

### Layout Settings in the Sample Container 18

All fed-batch cultivations took place in the sample container 18 (FIG. 8). Row A contained 1,900 μL of the glucose feed solution and row B was filled with 1,900 μL of the pH-adjusting agent. Software adjusted the pump volumes to 0.30 μL for aqueous solutions (3 M NaOH) and to 0.16 μL for the more viscous feed solution (500 g/L glucose).

In all fed-batch experiments, the feeding was time triggered and the feed profile was set to a constant feed with 4 μL/h. The pH control was set to pH 6.0. The anaerobic conditions during all cultivations in the sample container 18 were achieved by using the gassing lid, which was attached to the sample container 18 after it was prepared and sealed with the gas permeable sterile silicon foil (F-GPRSMF32-1).

### Results

Fed-batch Cultivation of Lactobacillus casei in the Microbioreactor

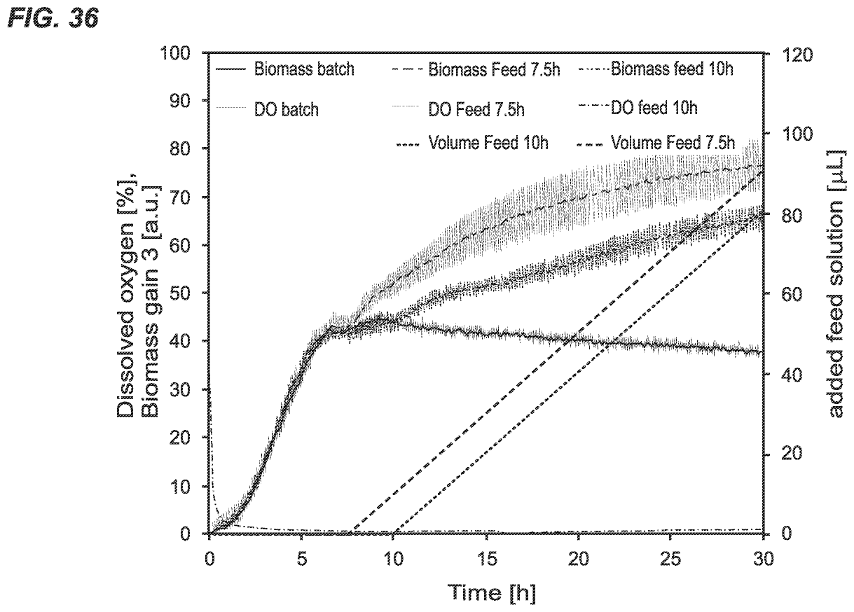

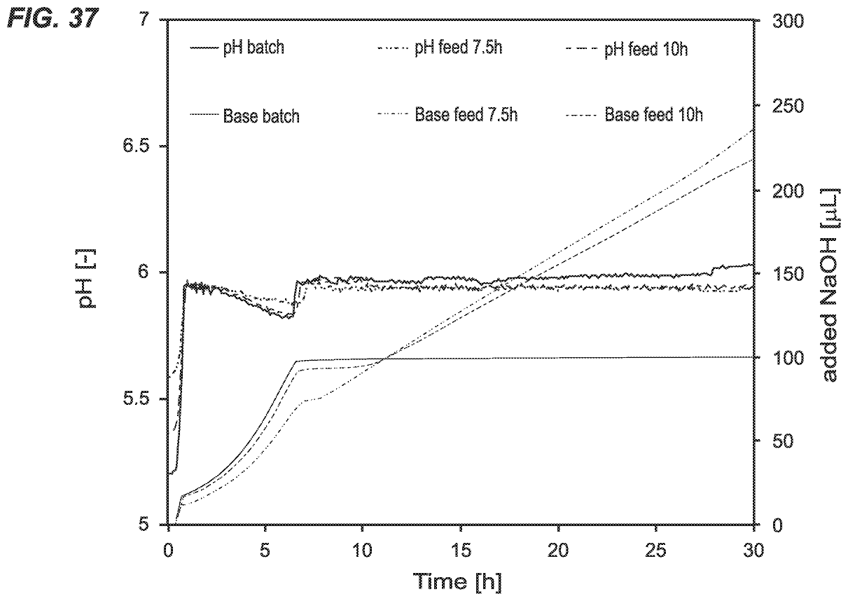

In FIGS. 36 and 37, the cultivation process of Lactobacillus casei in MRS broth is shown. In FIG. 36, the online signals of biomass and dissolved oxygen (DO) signal, and the volume of the added feed solution (500 g/L glucose) are presented. In FIG. 37, the online values of pH and the associated volumes of NaOH are plotted against cultivation time.

Here, three different process setups were applied: a batch cultivation and two fed-batch cultivations. One with a feed start after 7.5 hours and the other with a feed start after 10 hours. With a continuous flowrate of 30 mL/min N2, the DO decreased steadily. After 45 minutes, a DO below 5% was reached and decreased further. After 4.5 h the DO reached below 0.5% and continued to drop towards 0%. With the initiation of the stationary phase of the culture at around 6.7 hours, the exponential growth stops, and the biomass signal was 42 a.u. in all three culture approaches at this timepoint. The batch culture grows further slowly to a maximum of 44 a.u. at 9.5 hours then it steadily decreases to a final biomass signal of 38 a.u. at the end of cultivation. An increase in the biomass signal is correlated with the addition of the feed solution. As soon as the feed starts, an increase of the biomass signal is visible. The final biomass signal for the 7.5 h-fed-batch process was 76.3 a.u. and for the 10 h-fed-batch process, it led to a final biomass signal of 65.5 a.u. after 30 hours. The values for the added base solution are growth correlated. The addition of 3 M NaOH was stopped with the initiation of the stationary phase because no further bacterial acid production took place due to no growth. In the case of the constant addition feed solution, the acid production continued and thus, base was further needed to maintain the pH set point value of pH6.0.

This experiment shows that the container assembly 200 is a suitable device for anaerobic cultivations due to the gassing lid and the successful application of pH-control and feeding at the same time with direct anaerobic gassing.

### Technical and Biological Validation of the Anaerobic Conditions in the BioLector XT Device

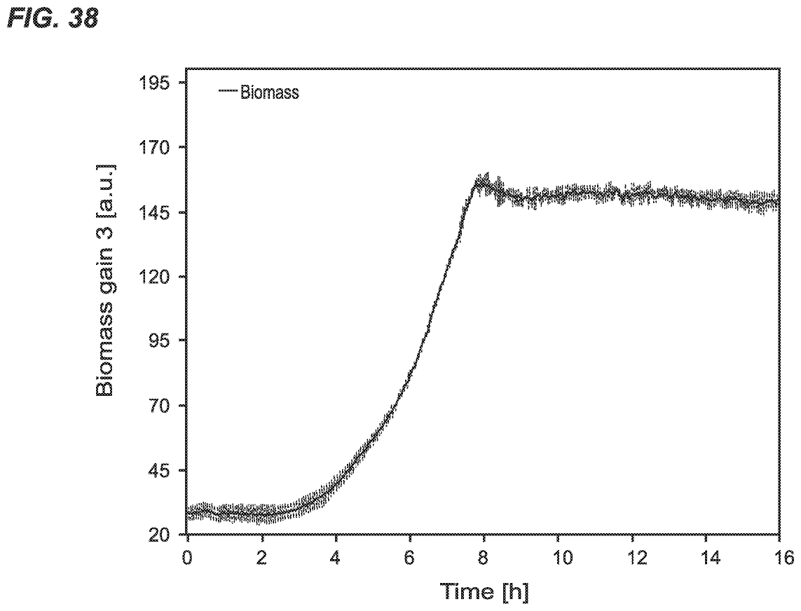

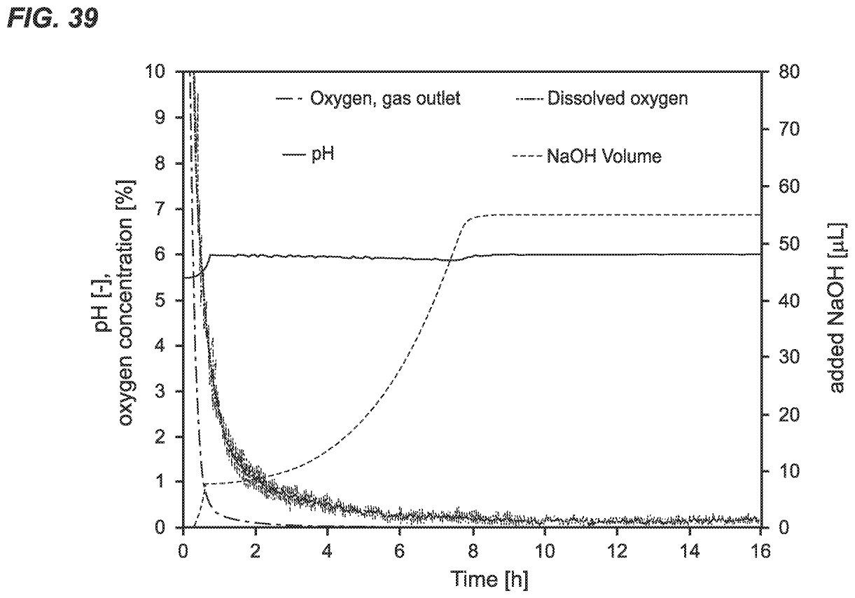

Maintaining anaerobic conditions during the whole cultivation time is an important requirement in case of the cultivation of oxygen sensitive organisms. In the following experiment, an external oxygen sensor was installed at the gas outlet of the container assembly 200 to validate the technical functionality of the gassing lid and to prove the tightness of the gassing lid and thus, the anaerobic atmosphere. In FIGS. 38 and 39, the experimental data of a batch cultivation of Lactobacillus plantarum (L. plantarum) are shown. In FIG. 38, the online biomass signal (gain 3) is shown. In FIG. 39, the online signal of dissolved oxygen in the culture broth and the oxygen concentration in the gas outlet of the container assembly 200, the online pH signal and the added NaOH volume for the pH-control are shown.

After a lag-time of 2.86 hours, the exponential growth started. The final biomass signal was 155.865 a.u. (OD600=9.01±0.07) after 7.96 hours when the stationary phase was initiated. During the growth of L. plantarum, lactic acid production took place. That acid formation growth is correlated to the added NaOH volume to maintain pH 6. With a continuous flowrate of 30 mL/min N2 the DO decreased steadily. After 39 min, a DO below 5% was reached and decreased further. After 4 hours, the DO dropped further below 0.5% and continued to drop towards 0%. The external sensor showed a final oxygen concentration of 0.029% after a cultivation time of 16 hours.

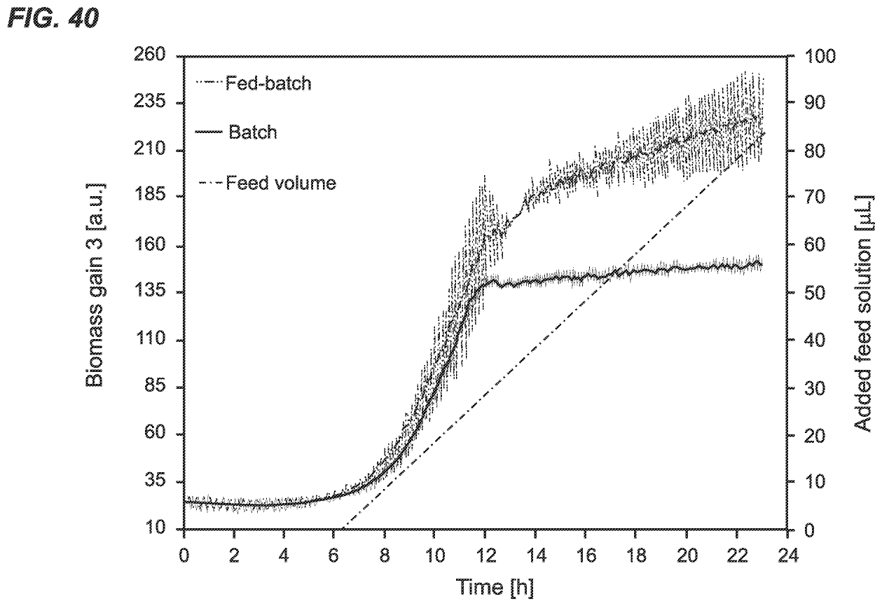

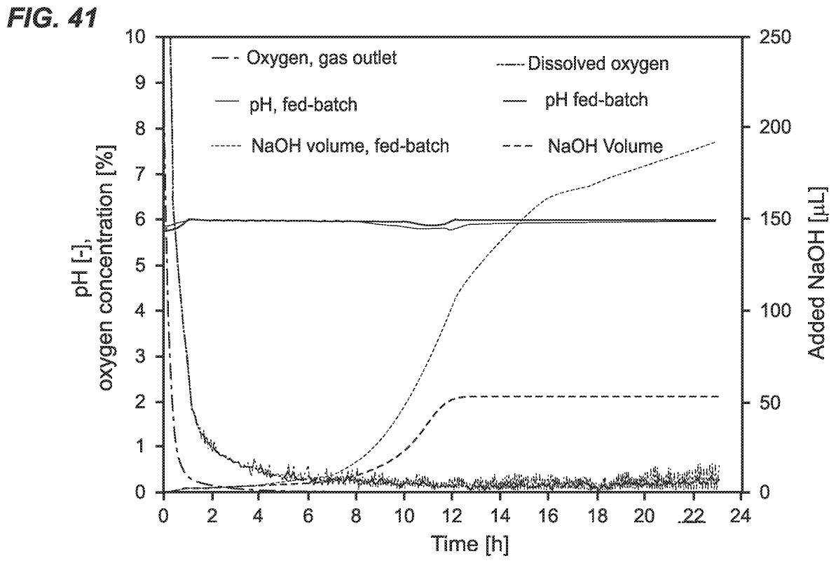

With this cultivation example, the technical functionality was validated, but the fact that Lactobacillus spp. can also grow under aerobic conditions and can even metabolize oxygen is not sufficient evidence for the biological validation of anaerobic cultivation in the container assembly 200. Therefore, the strict anaerobic Bifidobacterium bifidum was cultivated. The successful cultivation of this strain serves as the biological validation for anaerobic cultivations in the container assembly 200. In FIGS. 40 and 41, experimental data of a batch as well as a fed-batch cultivation of B. bifidum is shown. In FIG. 40, online signals of biomass and the added feed volume is plotted against the cultivation time. In FIG. 41, the online (optodes) signal of pH and DO, as well the added volume of 3 M NaOH and the oxygen signal of the external gas sensor in the gas outlet of the container assembly 200, are presented.

After a lag-time of 2.4 hours, the exponential growth started and for the batch culture the biomass signal reached a final value of 147.57 a.u. (OD600=8.3±0.57). In contrast to the batch culture, an extended exponential growth phase is observable. This phenomenon is caused by a higher amount of glucose in the medium as the feed already started after 6 hours. After 23 hours of cultivation, a maximum biomass value of 227.3 a.u. (OD600=15.93±0.69) was achieved. During the growth of B. bifidum, lactic acid production occurred and its growth correlated, which is observable in the curve of the addition of NaOH to maintain the pH at pH 6. In total, 193.56 μL of 3 M NaOH were pumped into the culture broth. With a continuous flowrate of 30 mL/min N2, the DO decreased steadily.

The external oxygen data already described for the first 16 hours since the cultivation of L. plantarum (as described earlier) and B. bifidum were gained simultaneously in the same container assembly 200 run and thus, the sample container 18, gassing lid, and external gas sensor were used. It is observable that the DO signal slightly increases from 18 hours, which could be explained by the technically conditioned signal drift of the oxygen optodes with a drift at 0% oxygen of <0.5% O2 per day. The data of the external oxygen sensor showed a value of 0.029% oxygen in the gas outlet of the container assembly 200 after 23 hours, confirming that the anaerobic cultivation conditions were maintained over the entire cultivation time.

In conclusion, a successfully conducted cultivation experiment of an anaerobic organism in the container assembly 200 is shown. In combination with microfluidic chip technology and the direct nitrogen gassing via the gassing lid, the simultaneous performance of pH control, feeding, and direct nitrogen gassing can be performed in small scale cultivations.

To sum up, the technical and biological validation of the cultivation of probiotics like Lactobacillus spp. and Bifidobacterium bifidum in the container assembly 200 in combination with the anaerobic gassing lid are shown. The microfluidic chip technology combined with the direct nitrogen gassing of the sample container 18 via the gassing lid enables the simultaneous performance of pH control, feeding and direct nitrogen gassing in small scale cultivation systems. It is a suitable system for the cultivation of anaerobic bacteria.

Additional aspects of the present disclosure are listed in the following clauses:

Clause 1. A lid assembly comprising: a lid housing having a top exterior surface and a bottom interior surface, the lid housing configured to cover a sample container; a first resilient layer disposed in the lid housing: and a sealing surface projecting from the bottom interior surface of the lid housing toward the first resilient layer to create an air-tight seal when the sealing surface is pressed against the first resilient layer.

Clause 2. The lid assembly of clause 1, the first resilient layer including one or more first apertures aligned with a respective guide element, each first aperture being configured to open when a pipette tip is pushed through and to close when the pipette tip is removed.

Clause 3. A lid assembly comprising: a lid housing having a top exterior surface and a bottom interior surface, the lid housing configured to cover a sample container: one or more guide elements extending from the top exterior surface of the lid housing, each guide element having a hollow interior portion running from a top end to a bottom end, the hollow interior portion having a larger cross-sectional area at the top end than at the bottom end, and each guide element being configured to receive and guide a pipette tip; and a first layer disposed in the lid housing, the first layer including one or more first apertures aligned with a respective guide element, each first aperture being configured to open when the pipette tip is pushed through and to close when the pipette tip is removed.

Clause 4. The lid assembly of clause 3, further comprising: a sealing surface projecting from the bottom interior surface of the lid housing toward the first layer to create an air-tight seal when the sealing surface is pressed against the first layer.

Clause 5. The lid assembly of clause 4, wherein the sealing surface includes a partition dividing a first recessed area on the bottom interior surface of the lid housing from a second recessed area on the bottom interior surface of the lid housing.

Clause 6. The lid assembly of clause 5, wherein the sealing surface and the partition are continuous with one another.

Clause 7. The lid assembly of clause 5, further comprising a first gas port connected to the first recessed area of the lid housing and configured to receive a pressurizing gas.

Clause 8. The lid assembly of clause 7, further comprising second and third gas ports configured to receive or remove one or more gases from the second recessed area.

Clause 9. The lid assembly of clause 5, further comprising: one or more additional partitions configured to separate additional recessed areas between the bottom interior surface of the lid housing and the first layer.

Clause 10. The lid assembly of clause 4, wherein the sealing surface is made of a rigid material.

Clause 11. The lid assembly of clause 4, wherein the sealing surface is made of PEEK.

Clause 12. The lid assembly of clause 3, further comprising: a sterile layer disposed on a bottom side of the first layer, wherein the sterile layer is configured to be pierced by the pipette tip.

Clause 13. The lid assembly of clause 3, further comprising: a second layer disposed between the bottom end of each guide element and the top exterior surface of the lid housing, the second layer having one or more second apertures aligned with respective guide elements and first apertures, and providing access to one or more through-holes in the lid housing, each second aperture being configured to open when the pipette tip is pushed through the second aperture and to close when the pipette tip is removed.

Clause 14. The lid assembly of clause 3, further comprising one or more posts extending from the bottom interior surface of the lid housing toward the first layer.

Clause 15. The lid assembly of clause 3, wherein the one or more guide elements form an integral part of the lid housing.

Clause 16. The lid assembly of clause 15, wherein the one or more guide elements are removably coupled to the top exterior surface of the lid housing.

Clause 17. The lid assembly of clause 15, wherein the hollow interior portions has a conical or frustoconical shape.

Clause 18. The lid assembly of clause 15, wherein the one or more first apertures are slits.

Clause 19. The lid assembly of clause 18, wherein the slits are self-healing.

Clause 20. The lid assembly of clause 3, wherein the first layer is a resilient polymer material.

Clause 21. The lid assembly of clause 3, wherein the first layer is made from silicone.

Clause 22. A container assembly comprising: a lid assembly comprising: a lid housing with a top exterior surface and a bottom interior surface, the lid housing configured to cover a sample container; one or more guide elements extending from the top exterior surface of the lid housing, each guide element having a hollow interior portion running from a top end to a bottom end, the hollow interior portion having a larger cross-sectional area at the top end than at the bottom end, and each guide element being configured to receive and guide a pipette tip; and a first layer disposed in the lid housing, the first layer having one or more first apertures aligned with respective guide elements, each first aperture being configured to open when the pipette tip is pushed through and to close when the pipette tip is removed; and a sample container comprising a plurality of wells.

Clause 23. The container assembly of clause 22, wherein a first portion of the sample container comprises one or more first wells and a second portion of the sample container comprises one or more second wells, wherein the one or more first wells are configured to contain fluid reagents and the one or more second wells are configured to contain a fluid sample comprising one or more cells, wherein one or more of the first wells are fluidically coupled to one or more of the second wells via one or more fluidic channels: wherein the lid assembly provides an air-tight seal around the sample container when the lid assembly is caused to be compressed against the sample container.

Clause 24. The container assembly of clause 23, wherein the air-tight seal has a first sealing surface on the sample container and a second sealing surface on the lid assembly pressing against the first sealing surface whereby both sealing surfaces act perpendicular to the bottom interior surface of the lid housing.

Clause 25. The container assembly of clause 23, further comprising: an eccentric lever and a ball sleeve comprising radially guided balls.

Clause 26. A bioreactor system comprising: a reversibly sealable sample container assembly comprising: a lid assembly comprising: a lid housing having a top exterior surface and a bottom interior surface, the lid housing configured to cover a sample container; one or more guide elements extending from the top exterior surface of the lid housing, each guide element having a hollow interior portion running from a top end to a bottom end, the hollow interior portion having a larger cross-sectional area at the top end than at the bottom end, and each guide element being configured to receive and guide a pipette tip; a first layer disposed in the lid housing, the first layer having one or more first apertures aligned with respective guide elements, each first aperture being configured to open when the pipette tip is pushed through and to close when the pipette tip is removed; a sample container comprising a plurality of wells; a platform configured to shake the sample container assembly by moving the sample container assembly within a predetermined range of motion, wherein the predetermined range of motion is within one or more interior diameters of one or more top ends of one or more of the guide elements: and pipetting robot having one or more pipette tips configured for insertion into the sample container via the one or more guide elements while the sample container assembly is being shaken.

Clause 27. The bioreactor system of clause 26, wherein the platform is configured to move the sample container assembly in an orbital fashion.

Clause 28. The bioreactor system of clause 26, wherein the platform is configured to move the sample container assembly in an orbital fashion within a range of 600 RPM to 1000 RPM.

Clause 29. The bioreactor system of clause 26, wherein the platform is configured to move the sample container assembly in an orbital fashion within a range of 600 RPM to 800 RPM.

Clause 30. The bioreactor system of clause 26, 28, or 29, wherein an agitation diameter of the orbital movement of the sample container assembly is within a range of 1 mm to 5 mm.

Clause 31. A method of sealing a sample container comprising: placing a sterile layer on top of the sample container; placing a resilient layer on top of the sterile layer; pressing a lid housing on top of the resilient layer; and releasably securing the lid housing to the sample container.

Clause 32. The method of clause 31, further comprising actuating an eccentric lever and ball sleeve comprising radially guided balls.

Clause 33. The method of clause 31, further comprising actuating release pins to release the sample container from the lid housing.

Clause 34. A method of cultivating anaerobic cells, the method comprising: placing a sample container within an anaerobic environment; disposing a sample comprising anaerobic cells into one or more wells of the sample container while the sample container is in the anaerobic environment; creating an air-tight seal around the wells of the sample container by placing a lid assembly over the wells of the sample container; and transporting the sealed sample container to a non-anaerobic environment for cell cultivation.

Clause 35. The method of clause 34, wherein the sealed sample container is placed within a microbioreactor disposed in the non-anaerobic environment.

Clause 36. The method of clause 34, wherein the sample container and lid assembly define a headspace above the one or more wells, and the method further comprises: adjusting an oxygen concentration in the headspace to between 0% and 5%.

Clause 37. The method of clause 34, wherein the sample container and lid assembly define a headspace above the one or more wells, and the method further comprises: adjusting an oxygen concentration in the headspace to between 0% and 10%.

Clause 38. The method of clause 34, wherein the sample container and lid assembly define a headspace above the one or more wells, and the method further comprises: adjusting an oxygen concentration in the headspace to between 0% and 20%.

Clause 39. A method of inserting a pipette tip into sample container while a bioreactor system is being shaken, the method comprising: placing a guide element above the sample container of the bioreactor system; shaking the bioreactor system; actuating a pipetting robot to guide the pipette tip to a narrowest region of the guide element; and guiding the pipette tip through the narrowest region of the guide element into the sample container.

Clause 40. The method of clause 39, further comprising removing a volume of fluid from the sample container via the pipette tip.

Clause 41. The method of clause 39, further comprising adding a volume of fluid to the sample container via the pipette tip.

Clause 42. A lid assembly for a microplate, wherein the microplate includes one or more wells, the lid assembly being configured to provide a headspace above the wells to allow gas exchange during cell cultivation, wherein the headspace above the wells is 20 mL to 400 ml.

Clause 43. The lid assembly of clause 42, wherein the headspace is 60 ml to 90 ml.

Clause 44. A method of controlling gas concentrations in a headspace above wells of a microplate, the method comprising: placing a lid assembly above the microplate, the microplate including one or more wells, the lid assembly configured to provide a headspace above the wells to allow gas exchange during cell cultivation, wherein the headspace above the reservoirs is 20 mL to 400 mL; and causing a gas to flow into the headspace.

Clause 45. The method of clause 44, further comprising: measuring a concentration of the gas; and adjusting the gas flow based on the measured concentration.

Clause 46. A control system for a sample container assembly with a gassing lid, the control system comprising: sensors configured to acquire measurement parameters associated with the sample container assembly; a gas supply system providing at least one gas to the gassing lid; and a controller configured to process the acquired measurement parameters and control the gas supply based upon the processed measurement parameters.

Clause 47. A method of controlling a sample container assembly with a gassing lid, the method comprising: sensing measurement parameters associated with the sample container assembly; processing the sensed measurement parameters; and controlling a gas supply of at least one gas to the gassing lid based upon the processed measurement parameters.

Clause 48. A computer program product, that stores in a tangible and non-transitory manner, a computer program code, that when executed by a computer controller, causes the computer controller to: sense measurement parameters associated with a sample container assembly having a gassing lid; process the sensed measurement parameters; and control a gas supply of at least one gas to the gassing lid based upon the processed measurement parameters.

Clause A1. A bioreactor system comprising: a lid assembly including: a lid housing having a top exterior surface and a bottom interior surface, the lid housing configured to cover a sample container; and a first layer disposed in the lid housing; and wherein the bottom interior surface includes a sealing surface projecting toward the first layer to create an air-tight seal when the sealing surface is pressed against the first layer.

Clause A2. The bioreactor system of claim 1, wherein the first layer includes one or more first apertures configured for alignment with a respective guide element, each first aperture being configured to open when a pipette tip is pushed through and to close when the pipette tip is removed.

Clause A3. The bioreactor system of clause A1, further comprising: one or more guide elements extending from the top exterior surface of the lid housing, each of the one or more guide elements having a hollow interior portion running from a top end to a bottom end, the hollow interior portion having a larger cross-sectional area at the top end than at the bottom end, and each of the one or more guide elements being configured to receive and guide a pipette tip; and wherein the first layer includes one or more first apertures each aligned with a respective guide element of the one or more guide elements, each first aperture being configured to open when the pipette tip is pushed through and to close when the pipette tip is removed.

Clause A4. The bioreactor system of clause A3, wherein the sealing surface includes a partition dividing a first recessed area on the bottom interior surface of the lid housing from a second recessed area on the bottom interior surface of the lid housing.

Clause A5. The bioreactor system of clause A4, wherein the sealing surface and the partition are continuous with one another.

Clause A6. The bioreactor system of clause A4, further comprising a first gas port connected to the first recessed area of the lid housing and configured to receive a pressurizing gas.

Clause A7. The bioreactor system of clause A6, further comprising a second gas port and a third gas port, wherein the second and third gas ports are configured to receive and/or remove one or more gases from the second recessed area.

Clause A8. The bioreactor system of clause A4, further comprising: one or more additional partitions configured to separate additional recessed areas between the bottom interior surface of the lid housing and the first layer.

Clause A9. The bioreactor system of clause A4, wherein the sealing surface is made of a rigid material.

Clause A10. The bioreactor system of clause A4, wherein the sealing surface is made of polyether ether ketone (PEEK).

Clause A11. The bioreactor system of clause A3, further comprising: a sterile layer disposed on a bottom side of the first layer, wherein the sterile layer is configured to be pierced by the pipette tip.

Clause A12. The bioreactor system of clause A3, further comprising: a second layer disposed between the bottom end of each of the one or more guide elements and the top exterior surface of the lid housing, the second layer having one or more second apertures aligned with a respective guide element of the one or more guide elements and a respective first aperture of the one or more first apertures, and providing access to a through-hole in the lid housing, each of the one or more second apertures being configured to open when the pipette tip is pushed through the second aperture and to close when the pipette tip is removed.

Clause A13. The bioreactor system of clause A3, further comprising one or more posts extending from the bottom interior surface of the lid housing toward the first layer.

Clause A14. The bioreactor system of clause A3, wherein the one or more guide elements form an integral part of the lid housing.

Clause A15. The bioreactor system of clause A3, wherein the one or more guide elements are removably coupled to the top exterior surface of the lid housing.

Clause A16. The bioreactor system of clause A3, wherein the hollow interior portion has a frustoconical shape.

Clause A17. The bioreactor system of clause A3, wherein the one or more first apertures are slits.

Clause A18. The bioreactor system of clause A3, wherein the one or more first apertures are self-healing.

Clause A19. The bioreactor system of clause A3, wherein the first layer is a resilient polymer material.

Clause A20. The bioreactor system of clause A3, wherein the first layer is made of silicone.

Clause A21. The bioreactor system of clause A3, further comprising: the sample container comprising a plurality of wells.

Clause A22. The bioreactor system of clause A21, wherein a first portion of the sample container includes one or more first wells and a second portion of the sample container includes one or more second wells, wherein the one or more first wells are configured to contain fluid reagents and the one or more second wells are configured to contain a fluid sample comprising one or more cells, wherein one or more of the first wells are fluidically coupled to one or more of the second wells via one or more fluidic channels; and wherein the lid assembly provides an air-tight seal around the sample container when the lid assembly is caused to be compressed against the sample container.

Clause A23. The bioreactor system of clause A22, wherein the air-tight seal has a first sealing surface on the sample container and a second sealing surface on the lid assembly pressing against the first sealing surface whereby both the first and second sealing surfaces act perpendicular to the bottom interior surface of the lid housing.

Clause A24. The bioreactor system of clause A22, further comprising: an eccentric lever and a ball sleeve comprising radially guided balls configured to compress the lid assembly against the sample container.

Clause A25. The bioreactor system of clause A21, further comprising: a platform configured to shake the sample container by moving the sample container within a predetermined range of motion, wherein the predetermined range of motion is defined as being less than an interior diameter of the top end of each of the one or more of the guide elements; and a pipetting robot having one or more pipette tips configured for insertion into the sample container via the one or more guide elements while the sample container assembly is being shaken.

Clause A26. The bioreactor system of clause A25, wherein the platform is configured to move the sample container in an orbital movement.

Clause A27. The bioreactor system of clause A25, wherein the platform is configured to move the sample container in an orbital movement within a range of 600 RPM to 1000 RPM.

Clause A28. The bioreactor system of clause A25, wherein the platform is configured to move the sample container in an orbital movement within a range of 600 RPM to 800 RPM.

Clause A29. The bioreactor system of clause A26, 27, or 28, wherein an agitation diameter of the orbital movement of the sample container is within a range of 1 mm to 5 mm.

Clause A30. The bioreactor system of clause A21, further comprising: sensors configured to acquire measurement parameters associated with the sample container; a gas supply system providing at least one gas to the lid assembly; and a controller configured to process the measurement parameters and control the gas supply based upon the measurement parameters.

Clause A31. A method of cultivating cells, the method comprising: placing a sterile layer on top of a sample container; placing a first layer on top of the sterile layer; pressing a lid housing on top of the first layer; and releasably securing the lid housing to the sample container.

Clause A32. The method of clause A31, further comprising: actuating an eccentric lever and ball sleeve comprising radially guided balls to releasably secure the lid housing to the sample container.

Clause A33. The method of clause A31, further comprising: actuating release pins to release the sample container from the lid housing.

Clause A34. The method of clause A31, further comprising: placing the sample container within an anaerobic environment; disposing a sample comprising anaerobic cells into one or more wells of the sample container while the sample container is in the anaerobic environment; creating an air-tight seal around the one or more wells of the sample container by placing the lid housing over the one or more wells of the sample container; and transporting the sample container to a non-anaerobic environment for cell cultivation.

Clause A35. The method of clause A34, further comprising: placing the sample container within a microbioreactor disposed in the non-anaerobic environment.

Clause A36. The method of clause A34, wherein the sample container and the lid housing define a headspace above the one or more wells, and the method further comprises: adjusting an oxygen concentration in the headspace to be between 0% and 5%.

Clause A37. The method of clause A34, wherein the sample container and the lid housing define a headspace above the one or more wells, and the method further comprises: adjusting an oxygen concentration in the headspace to be between 0% and 10%.

Clause A38. The method of clause A34, wherein the sample container and the lid housing define a headspace above the one or more wells, and the method further comprises: adjusting an oxygen concentration in the headspace to be between 0% and 20%.

Clause A39. The method of clause A35, further comprising: inserting a pipette tip into the sample container while the sample container is being shaken by the microbioreactor.

Clause A40. The method of clause A39, further comprising: actuating a pipetting robot to guide the pipette tip to a narrowest region of a guide element placed above the lid housing; and guiding the pipette tip through the narrowest region of the guide element into the sample container.

Clause A41. The method of clause A40, further comprising: removing a volume of fluid from the sample container via the pipette tip.

Clause A42. The method of clause A40, further comprising: adding a volume of fluid to the sample container via the pipette tip.

Clause A43. The method of clause A34, wherein the lid housing provides a headspace above the one or more wells allowing for gas exchange during cell cultivation, and wherein the headspace above the one or more wells is 20 mL to 400 ml.

Clause A44. The method of clause A43, wherein the headspace above the one or more wells is 60 ml to 90 ml.

Clause A45. The method of clause A43, further comprising: causing a gas flow into the headspace: measuring a concentration of the gas in the headspace; and adjusting the gas flow based on the concentration.

Clause A46. The method of clause A43, further comprising: sensing a parameter in the sample container; processing the parameter; and controlling a gas supply of at least one gas to the lid housing based upon the processing of the parameter.

While the invention has been illustrated and described in connection with currently preferred embodiments shown and described in detail, it is not intended to be limited to the details shown since various modifications and structural changes may be made without departing in any way from the spirit and scope of the present invention. The embodiments were chosen and described in order to explain the principles of the invention and practical application to thereby enable a person skilled in the art to best utilize the invention and various embodiments with various modifications as are suited to the particular use contemplated.

1. A bioreactor system comprising:
a lid assembly including:
a lid housing having a top exterior surface and a bottom interior surface, the lid housing configured to cover a sample container; and
a first layer disposed in the lid housing; and
wherein the bottom interior surface includes a sealing surface projecting toward the first layer to create an air-tight seal when the sealing surface is pressed against the first layer.

2. The bioreactor system of claim 1, wherein the first layer includes one or more first apertures configured for alignment with a respective guide element, each first aperture being configured to open when a pipette tip is pushed through and to close when the pipette tip is removed.

3. The bioreactor system of claim 1, further comprising:
one or more guide elements extending from the top exterior surface of the lid housing, each of the one or more guide elements having a hollow interior portion running from a top end to a bottom end, the hollow interior portion having a larger cross-sectional area at the top end than at the bottom end, and each of the one or more guide elements being configured to receive and guide a pipette tip; and
wherein the first layer includes one or more first apertures each aligned with a respective guide element of the one or more guide elements, each first aperture being configured to open when the pipette tip is pushed through and to close when the pipette tip is removed.

4. The bioreactor system of claim 3, wherein the sealing surface includes a partition dividing a first recessed area on the bottom interior surface of the lid housing from a second recessed area on the bottom interior surface of the lid housing.

5. The bioreactor system of claim 4, wherein the sealing surface and the partition are continuous with one another.

6. The bioreactor system of claim 4, further comprising a first gas port connected to the first recessed area of the lid housing and configured to receive a pressurizing gas.

7. The bioreactor system of claim 6, further comprising a second gas port and a third gas port, wherein the second and third gas ports are configured to receive and/or remove one or more gases from the second recessed area.

8. The bioreactor system of claim 4, further comprising:
one or more additional partitions configured to separate additional recessed areas between the bottom interior surface of the lid housing and the first layer.

9-10. (canceled)

11. The bioreactor system of claim 3, further comprising:
a sterile layer disposed on a bottom side of the first layer, wherein the sterile layer is configured to be pierced by the pipette tip.

12. The bioreactor system of claim 3, further comprising:
a second layer disposed between the bottom end of each of the one or more guide elements and the top exterior surface of the lid housing, the second layer having one or more second apertures aligned with a respective guide element of the one or more guide elements and a respective first aperture of the one or more first apertures, and providing access to a through-hole in the lid housing, each of the one or more second apertures being configured to open when the pipette tip is pushed through the second aperture and to close when the pipette tip is removed.

13. The bioreactor system of claim 3, further comprising one or more posts extending from the bottom interior surface of the lid housing toward the first layer.

14. The bioreactor system of claim 3, wherein the one or more guide elements form an integral part of the lid housing.

15. The bioreactor system of claim 3, wherein the one or more guide elements are removably coupled to the top exterior surface of the lid housing.

16. The bioreactor system of claim 3, wherein the hollow interior portion has a frustoconical shape.

17. The bioreactor system of claim 3, wherein the one or more first apertures are slits.

18. The bioreactor system of claim 3, wherein the one or more first apertures are self-healing.

19-20. (canceled)

21. The bioreactor system of claim 3, further comprising:
the sample container comprising a plurality of wells.

22. The bioreactor system of claim 21, wherein a first portion of the sample container includes one or more first wells and a second portion of the sample container includes one or more second wells, wherein the one or more first wells are configured to contain fluid reagents and the one or more second wells are configured to contain a fluid sample comprising one or more cells, wherein one or more of the first wells are fluidically coupled to one or more of the second wells via one or more fluidic channels; and
wherein the lid assembly provides an air-tight seal around the sample container when the lid assembly is caused to be compressed against the sample container.

23. The bioreactor system of claim 22, wherein the air-tight seal has a first sealing surface on the sample container and a second sealing surface on the lid assembly pressing against the first sealing surface whereby both the first and second sealing surfaces act perpendicular to the bottom interior surface of the lid housing.

24. The bioreactor system of claim 22, further comprising:
an eccentric lever and a ball sleeve comprising radially guided balls configured to compress the lid assembly against the sample container.

25-46. (canceled)
